(19) United States
(12) Patent Application Publication
(10) Pub. No.: US 2026/0115349 A1
(43) Pub. Date: Apr. 30, 2026

(54) Long-Lasting Antimicrobial Surfaces Based On The Cross-Linking Of Natural Oils Within Polymer Networks

(71) Applicant: The Regents of The University of Michigan, Ann Arbor, MI (US)

(72) Inventors: Anish TUTEJA, Ann Arbor, MI (US); Sarah SNYDER, Ann Arbor, MI (US); Abhishek DHYANI, Ann Arbor, MI (US); Kevin GOLOVIN, Kelowna (CA); Jeremy Scott VANEPPS, Ann Arbor, MI (US); Geeta MEHTA, Ann Arbor, MI (US)

(73) Assignee: The Regents of The University of Michigan, Ann Arbor, MI (US)

(21) Appl. No.: 19/003,605

(22) Filed: Dec. 27, 2024

Related U.S. Application Data
(62) Division of application No. 17/054,975, now abandoned.
(60) Provisional application No. 62/671,060, filed on May 14, 2018.

(51) Int. Cl. A61L 15/20; A61L 15/24; A61L 15/26; A61L 15/44; A61L 15/46; A61L 27/26; A61L 27/36; A61L 27/54

(52) U.S. Cl. CPC A61L 15/20; A61L 15/24; A61L 15/26; A61L 15/44; A61L 15/46; A61L 27/26; A61L 27/3637; A61L 27/54; A61L 2300/30; A61L 2300/404

(57) ABSTRACT

An antimicrobial composition is provided. The antimicrobial composition includes a polymer matrix, an oil-derived component covalently bonded to the polymer matrix, and an oil-derived antimicrobial component non-covalently associated with at least one of the polymer matrix and the oil-derived component. Methods of making and using the antimicrobial composition are also provided.

### CROSS-REFERENCE TO RELATED APPLICATIONS

This application is a divisional application of U.S. Non-Provisional application Ser. No. 17/054,975, filed Nov. 12, 2020, which is a National Stage of International Application No. PCT/US2019/032173, filed May 14, 2019, which claims the benefit of U.S. Provisional Application No. 62/671,060, filed May 14, 2018. The entire disclosures of each of the above applications are incorporated herein by reference.

### FIELD

The present disclosure relates to long-lasting antimicrobial surfaces based on the cross-linking of natural oils within polymer networks.

### BACKGROUND

This section provides background information related to the present disclosure which is not necessarily prior art.

Antifouling and antibacterial surfaces are of extreme interest due to a plethora of potential applications, such as saving lives with medical devices, preventing hospital acquired infections, and even preventing marine biofouling. Currently, there is no durable surface that can completely resist bio-adhesion from a variety of biomolecules for an extended period of time.

To create antifouling surfaces, much research has gone into testing surfaces that will repel the attachment of microbes. In particular, surfaces with different wettabilities have been investigated, as microbes preferentially adhere to hydrophobic surfaces due to cell proteins being comprised of long, hydrophobic carbon chains. Therefore, the approach of making antifouling surfaces out of hydrophilic surfaces has been extensively studied with polyethylene glycol (PEG) and zwitterionic polymer surfaces. Superhydrophilic surfaces, i.e., surfaces with approximately a 0° contact angle with water, have been shown to repel proteins better than hydrophilic surfaces. However, these hydrophilic or superhydrophilic surfaces can degrade, and/or they can become more hydrophobic over time, allowing microbes to eventually attach.

Achieving a long effective lifetime of antifouling surfaces continues to be a challenge, and several different approaches involving extreme wettabilities have been developed. The first approach involves incorporating scales of roughness onto a hydrophobic polymer surface. With surface roughness, surfaces are in the Cassie-Baxter or composite state, and thus, microbes are effectively in contact with only a fraction of the surface, with the liquid sitting on many tiny air bubbles. Several studies have investigated the antifouling effects of microstructured polydimethylsiloxane (PDMS) surfaces, polystyrene and polylactic acid composite surfaces, and nano-rough polysiloxane surfaces. Other techniques achieve superhydrophobicity and omniphobicity by combining different approaches. Slippery liquid-infused porous surfaces (SLIPs), for example, are tethered polymer surfaces infused with a fluorinated or non-fluorinated oil, so that a droplet in contact with the surfaces is only in contact with the infused oil. Another technique utilizes an amphiphilic block copolymer design based on polystyrene and polyacrylate blocks, and an additional method coats silica nanoparticles onto precipitated polymer spheres to get hierarchal microgel spheres that are then re-coated with a hydrophilic polymer. However, all of these approaches focus on eliminating biomolecule attachment and have no cytotoxic components; microbes are simply relocated elsewhere and still persist in the environment. Additionally, although textured hydrophobic surfaces initially reduce microbial attachment, bacteria still manage to overcome unfavorable surface topographies and attach to the entire surface. Finally, the abrasion resistance of these surfaces is generally poor, rendering them ineffective as surfaces that possess persistent (e.g., greater than 1 month) antimicrobial properties in real-life conditions.

Utilizing a cytotoxic component is a much simpler approach for antimicrobial activity. The properties of essential oils have been explored in a number of ways, as it is well known that many essential oils possess antibacterial properties. Unfortunately, although essential oils are antimicrobial, they are extremely volatile and evaporate very quickly. The most common solution to the volatility issue is to use some form of encapsulation. Some systems utilize a surfactant to maintain a microemulsion of essential oils in an aqueous phase for cleaners and disinfectants. Other systems utilize sodium alginate to encapsulate the essential oils for applications such as wound dressing. Another solution to extreme volatility is to use physical methods to immobilize the molecules of the essential oils and to limit their evaporation. These methods include oils coated onto nanoparticles, oils mixed into a polymeric network via an extruder, and adding a fragrance into a polyurethane foam for air freshening applications. However, since these methods only physically immobilize the molecules of the essential oils, it is just a matter of time before all the molecules evaporate and the antimicrobial activity ceases to exist.

Accordingly, the development of long-lasting antimicrobial surfaces is desired.

### SUMMARY

This section provides a general summary of the disclosure, and is not a comprehensive disclosure of its full scope or all of its features.

In various aspects, the current technology provides an antimicrobial composition including a polymer matrix, an oil-derived component covalently bonded to the polymer matrix, and an oil-derived antimicrobial component non-covalently associated with at least one of the polymer matrix and the oil-derived component.

In one aspect, the oil-derived antimicrobial component is physically associated with the at least one of the polymer matrix and the oil-derived component.

In one aspect, the oil-derived component and the oil-derived antimicrobial component are components of a plant oil extract.

In one aspect, the oil-derived component and the oil-derived antimicrobial component are components of an oil selected from the group including basil oil, bergamot oil, black pepper oil, Brazil's spearmint oil, cardamom oil, cedar oil, cinnamon oil, citron oil, clary sage oil, clove oil, coriander oil, cypress oil, eucalyptus oil, fennel oil, geranium oil, ginger oil, lavender oil, lemongrass oil, mandarin oil, marjoram oil, nutmeg oil, orange oil, oregano oil, palmarosa oil, patchouli oil, peppermint oil, perilla oil, pine oil, rosemary oil, Tahiti lime oil, tea tree oil, thyme oil, vetiver oil, ylang ylang oil, Achillea clavennae, Achillea fragrantissima, Achillea, Achillea ligustica, Artemisia absinthium, Artemisia biennis, Artemisia cana, Artemisia dracunculus, Artemisia longifolia, Artemisia frigida, Cinnamomum zeylancium, Copaifera officinalis, Cuminum cyminum, Cymbopogon citratus, Cymbopogon nardus, Cyperus longus, Daucus littoralis, Dracocephalum foetidum, Eremanthus erythropapps, Eugenia caryophyllata, Euphrasia rostkoviana, Fortunella margarita, Juniperus phoenicea, Laurus nobilis, Juniperus excelsa, Lippia sidoides, Mentha pulegium, Mentha suaveolens, Momordica charantia, Myrtus communis, Nigella sativa, Ocimum gratissimum, Ocimum kilimandscharicum, Origanum vulgare, Ocimum basilicum, Petroselinum sativum, Piper nigrum, Pimpinella anisum, Plectranthus barbatus, P. amboinicus, Plectranthus neochilus, Pogostemon cablin, Rosmarinus officinalis, Satureja hortensis, Salvia officinalis, Salvia lavandulifolia, Satureja cuneifolia, Struchium sparganophora, Syzygium cumini, Trachyspermum ammi, Thymus zygis, Thymus mastichina, Thymus kotschyanus, Thuja sp. (Thuja plicata, Thuja occidentalis), Verbena officinalis, Warionia saharae, fractions thereof, components thereof, molecules thereof, and combinations thereof.

In one aspect, the oil is tea tree oil, eucalyptus oil, or cinnamon oil.

In one aspect, the polymer matrix includes a polymer selected from the group including polyurethane, polyethers, polycarbonates, polyaspartics, polyesters, polyolefin, acrylates, poly(acrylic acid) (PAA), poly(methyl acrylate) (PMA), poly(methyl methacrylate) (PMMA), acrylonitrile butadiene styrene (ABS), polyamides, polylactic acid (PLA), polybenzimidazole, polycarbonate, polyether sulfone (PES), polyetherether ketone (PEEK), polyetherimide (PEI), polyethylene (PE), polyphenylene oxide (PPO), polyphenylene sulfide (PPS), polypropylene (PP), polystyrene (PS), polyvinyl chloride (PVC), polytetrafluoroethylene (PTFE), polyimides, vinyl esters, epoxy, polydimethylsiloxane, polyurethane (PU), perfluoropolyether (PFPE), polymethylhydrosiloxane (PMHS), polymethylphenylsiloxane (PMPS), copolymers of isocyanate functionalized polydimethylsiloxane (PDMS) and fluorinated polyurethane (FPU), copolymers of isocyanate functionalized polydimethylsiloxane (PDMS) and polyurethane (PU), acrylates, methacrylates, soybean oil acrylate, polystyrene, natural rubber, vulcanized rubber, synthetic rubber, butyl rubber, latex rubber, polychloroprene, acrylonitrile butadiene rubber, styrene butadiene rubber, elastomers made from ethylene propylene diene monomer (EPDM), epichlorohydrin-based rubber, poly(lactic-co-glycolic acid) (PLGA), epoxy, organogels, hydrogels, other elastomers, copolymers thereof, and combinations thereof.

In one aspect, the oil-derived component has antimicrobial activity.

In one aspect, the oil-derived component does not have antimicrobial activity.

In one aspect, the oil-derived component and the oil-derived antimicrobial component include the same oil-derived antimicrobial molecules.

In one aspect, the oil-derived component has molecules from an antimicrobial oil, and the oil-derived antimicrobial component includes the antimicrobial oil non-covalently associated within the polymer matrix.

In one aspect, the oil-derived component and the oil-derived antimicrobial component have a combined concentration in the antimicrobial composition of greater than or equal to about 1 wt. % to less than or equal to about 95 wt. %.

In one aspect, the oil-derived component and the oil-derived antimicrobial component are present in an oil-derived component:oil-derived antimicrobial component ratio of from about 1:100 to about 100:1.

In one aspect, the antimicrobial composition retains antimicrobial activity for a time period of greater than or equal to about 1 week.

In one aspect, the antimicrobial composition includes greater than or equal to about 33% to less than or equal to about 66% of the oil-derived component, with the remainder being the oil-derived antimicrobial component, and the antimicrobial composition retains antimicrobial activity for a time period of greater than or equal to about 3 months.

In one aspect, the antimicrobial composition kills greater than or equal to about 50% of bacteria, viruses, and fungi that contact the antimicrobial composition in a time period of less than or equal to about 45 minutes.

In one aspect, the antimicrobial composition is in the form of a solid film or coating.

In one aspect, the solid film or coating is textured.

In one aspect, the solid film is elastomeric and transparent, with a visible light transmission of greater than or equal to about 50%.

In one aspect, the solid film has an adhesive surface.

In one aspect, the current technology provides a wound dressing having a surface including the antimicrobial composition.

In one aspect, the wound dressing is less adhesive to a wound than a second dressing having the same dressing material, but without the antimicrobial composition.

In one aspect, the current technology provides a medical implant having a surface including the antimicrobial composition.

In one aspect, the current technology provides a high-touch surface including the antimicrobial composition, wherein the high-touch surface is selected from the group including a counter, a toilet, a sink, flooring, tiles, a dashboard, a handhold, a handle, a door handle, a door knob, a handrail, a cup holder, a touch screen, a tray, a tray table, furniture, paint, a table, a chair, a seat, a fabric, a gear shifter, and a steering wheel.

In one aspect, the current technology provides a medical implant including the antimicrobial composition.

In various aspects, the current technology further provides a method for generating an antimicrobial composition, the method including combining an antimicrobial oil or oil-derived antimicrobial molecules with an uncured polymer precursor solution to form a mixture and curing the mixture to generate the antimicrobial composition, wherein the antimicrobial composition includes a polymer matrix formed from the uncured polymer precursor solution, an oil-derived component covalently bonded to the polymer matrix, and an oil-derived antimicrobial component non-covalently associated with at least one of the polymer matrix and the oil-derived component, wherein the oil-derived component and the oil-derived antimicrobial component are provided from the antimicrobial oil or the oil-derived antimicrobial molecules.

In one aspect, the antimicrobial oil is selected from the group including basil oil, bergamot oil, black pepper oil, Brazil's spearmint oil, cardamom oil, cedar oil, cinnamon oil, citron oil, clary sage oil, clove oil, coriander oil, cypress oil, eucalyptus oil, fennel oil, geranium oil, ginger oil, lavender oil, lemongrass oil, mandarin oil, marjoram oil, nutmeg oil, orange oil, oregano oil, palmarosa oil, patchouli oil, peppermint oil, perilla oil, pine oil, rosemary oil, Tahiti lime oil, tea tree oil, thyme oil, vetiver oil, ylang ylang oil, Achillea clavennae, Achillea fragrantissima, Achillea, Achillea ligustica, Artemisia absinthium, Artemisia biennis, Artemisia cana, Artemisia dracunculus, Artemisia longifolia, Artemisia frigida, Cinnamomum zeylancium, Copaifera officinalis, Cuminum cyminum, Cymbopogon citratus, Cymbopogon nardus, Cyperus longus, Daucus littoralis, Dracocephalum foetidum, Eremanthus erythropapps, Eugenia caryophyllata, Euphrasia rostkoviana, Fortunella margarita, Juniperus phoenicea, Laurus nobilis, Juniperus excelsa, Lippia sidoides, Mentha pulegium, Mentha suaveolens, Momordica charantia, Myrtus communis, Nigella sativa, Ocimum gratissimum, Ocimum kilimandscharicum, Origanum vulgare, Ocimum basilicum, Petroselinum sativum, Piper nigrum, Pimpinella anisum, Plectranthus barbatus, P. amboinicus, Plectranthus neochilus, Pogostemon cablin, Rosmarinus officinalis, Satureja hortensis, Salvia officinalis, Salvia lavandulifolia, Satureja cuneifolia, Struchium sparganophora, Syzygium cumini, Trachyspermum ammi, Thymus zygis, Thymus mastichina, Thymus kotschyanus, Thuja sp. (Thuja plicata, Thuja occidentalis), Verbena officinalis, Warionia saharae, fractions thereof, components thereof, molecules thereof, and combinations thereof.

In one aspect, the antimicrobial oil is tea tree oil, eucalyptus oil, or a combination thereof.

In one aspect, the oil-derived antimicrobial molecules are selected from the group including alkaloids, glycosides, terpenes, terpenoids, isoprenoids, saponins, steroids, flavonoids, isoflavonoids, phenolics, polyphenols, phenylpropanoids, phenylpropenes, coumarins, curcuminoids, and combinations thereof.

In one aspect, the uncured polymer precursor solution includes at least one monomer.

In one aspect, the curing includes covalently bonding the oil-derived component to a portion of the at least one monomer and polymerizing a remaining portion of the at least one monomer to form the polymer matrix with the oil-derived component covalently bonded thereto.

In one aspect, the uncured polymer precursor solution includes either diisocyanate or dicarboxylic acid and polyol.

In one aspect, wherein the antimicrobial composition is a film and the method further includes disposing an adhesive onto a surface of the film.

In one aspect, the method is performed on a high-touch surface.

In one aspect, the method is performed on a medical implant.

In one aspect, the method further includes disposing a wound dressing into the mixture and performing the curing while the wound dressing is disposed in the mixture, wherein, after the curing, an antimicrobial wound dressing including the antimicrobial composition is formed.

In one aspect, the mixture includes greater than or equal to about 1 wt. % to less than or equal to about 95 wt. % of the antimicrobial oil.

In one aspect, the method further includes selecting the antimicrobial oil and the uncured polymer precursor solution such that the antimicrobial composition has a desired potency or effective duration.

In various aspects, the current technology additionally provides a method of preparing an antimicrobial surface, the method including applying a mixture onto a surface, the mixture having an antimicrobial oil or oil-derived antimicrobial molecules and an uncured polymer precursor solution, and incubating the mixture on the surface until the mixture cures and forms an antimicrobial film on the surface, the antimicrobial film including a polymer matrix formed from the uncured polymer precursor solution, an oil-derived component covalently bonded to the polymer matrix, and an oil-derived antimicrobial component non-covalently associated with at least one of the polymer matrix and the oil-derived component, wherein the oil-derived component and the oil-derived antimicrobial component are provided from the antimicrobial oil or the oil-derived antimicrobial molecules.

In one aspect, the antimicrobial oil is a natural oil extracted from a plant.

In one aspect, the mixture is composed from a kit including at least one uncured monomer, the antimicrobial oil or the oil-derived antimicrobial molecules, and, optionally, at least one of an initiator and an activator.

In one aspect, the antimicrobial film has a thickness of greater than or equal to about 1 μm to less than or equal to about 10 mm.

In one aspect, the surface on which the mixture is applied is a high-touch surface selected from the group including a counter, a toilet, a sink, flooring, tiles, a dashboard, a handhold, a handle, a door handle, a door knob, a handrail, a cup holder, a touch screen, a tray, a tray table, furniture, paint, a table, a chair, a seat, a fabric, a gear shifter, and a steering wheel.

In one aspect, the surface on which the mixture is applied is a surface of a medical implant or a surface of a wound dressing.

In one aspect, the current technology provides a method of rejuvenating an antimicrobial surface prepared by the method, the method including applying a fresh antimicrobial oil to the antimicrobial surface and incubating the antimicrobial surface until the fresh antimicrobial oil becomes physically associated with at least one of the antimicrobial film and the polymer matrix.

In various aspects, the current technology further provides a method for generating an antimicrobial composition, the method including combining a non-antimicrobial oil with an uncured polymer precursor solution to form a mixture, curing the mixture to generate a hardened composition, and contacting the hardened composition with an antimicrobial oil to form the antimicrobial composition.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an illustration of an antimicrobial composition according to various aspects of the current technology.

FIG. 2A is an illustration of the antimicrobial composition of FIG. 1 disposed on a first substrate according to various aspects of the current technology.

FIG. 2B is an illustration of the antimicrobial composition of FIG. 1 disposed on a second substrate according to various aspects of the current technology.

FIG. 3 is an illustration of the antimicrobial composition of FIG. 1, wherein the antimicrobial composition has an adhesive surface according to various aspects of the current technology.

FIG. 4A is a photograph of an antimicrobial wound dressing according to various aspects of the current technology.

FIG. 4B is a photograph of a wound dressing being removed from an artificial wound according to various aspects of the current technology.

FIG. 5 is a schematic showing free oil (black circles) stabilized by oil cross-linked (white triangles) into the cross-linkable polymer (polyurethane in this case) network (black lines). While some free oil remains in the bulk, it is shown that most assembles onto the surface.

FIG. 6 shows thermogravimetric analysis (TGA) data of DESMOPHEN® polyurethane (PU), DESMOPHEN® PU reacted with 30% tea tree oil (TTO), and DESMOPHEN® PU swelled in TTO at the 200° C. isotherm. DESMOPHEN® PU loses approximately 2 wt. %, while DESMOPHEN® PU reacted with TTO loses approximately 12 wt. %, indicating the presence of approximately 10 wt. % of free oil in the reacted samples. The DESMOPHEN® PU simply swelled with TTO loses approximately 29 wt. %, and when compared to the DESMOPHEN® PU reacted with TTO sample, the higher weight loss percentage is attributed to the lack of TTO stability within the DESMOPHEN® PU network, both chemically and physically. Thus, reacting the TTO with the DESMOPHEN® PU instead of simply swelling the PU fabricates a more stable PU+TTO network.

FIG. 7 is a bar graph representation of the adhered bacteria data for various surfaces and various surface testing conditions with both E. coli and S. aureus. All PU surfaces reacted with 30% TTO show at least an approximately 2.4-log reduction of adhered bacteria when compared to the PS and PU controls, with the fresh PU+30% TTO samples showing a 99.8% and 99.9% reduction of adhered bacteria with E. coli and S. aureus, respectively, when compared to the DESMOPHEN® PU. Results are similar with the abraded samples (99.6% and 99.9% of adhered E. coli and S. aureus, respectively, when compared to the DESMOPHEN® PU), demonstrating the physical durability of the surface. Even after 8 and 12 weeks of exposure in a chemical fume hood, the PU+30% TTO samples show a significant reduction in adhered bacteria—at least 99% for both E. coli and S. aureus when compared to the DESMOPHEN® PU. In comparison, while the Epoxy+30% TTO and PDMS+30% TTO surfaces initially show an approximately 2-log reduction in adhered bacteria, the Epoxy+30% TTO, 2 weeks and PDMS+30% TTO, 2 weeks surfaces show significant fouling. This is attributed to the fact that tea tree oil does not chemically cross-link into epoxy and PDMS, and therefore, the free oil is not stabilized in the polymeric network.

FIG. 8 shows ISO 22196 test results as performed by Microchem Laboratory. These results indicate a 99.998% and a greater than 99.995% reduction for E. coli and S. aureus, respectively.

FIG. 9 shows contact plate experiments for determining the time taken for the polyurethane cross-linked with tea tree oil surface to kill S. aureus bacteria. This shows the total number of colonies of S aureus growing on an Agar plate after 100,000 colonies of the bacteria come in contact with a polystyrene surface, a polyurethane surface, or the same polyurethane surface cross-linked with tea tree oil for 10 minutes.

FIG. 10 is a graph showing bacterial growth on common surfaces. In particular, the graph shows growth of MRSA and E. Coli (UTI189) on glass, polystyrene (PS), polyurethane (PU), and stainless steel (SS). The initial inoculum is 1 μmillion CFUs, which is depicted by the dotted line. The samples are tested via broth culture for over 24 hours at 37° C. inside an orbital shaker (200 RPM). Error bars indicate one standard deviation.

FIG. 11 is a graph showing results of durability testing of an antimicrobial coating prepared in accordance with the current technology (DESMOPHEN® polyurethane polymer matrix and 35 wt. % alpha (α)-terpineol). The coating is subjected to different durability tests, which include 500 cycles of CLOROX® disinfecting wipes, 1000 cycles of linear Taber abrasion, exposure to −17° C. for 25 hours, exposure to 254 nm UVC, and air flow exposure for a duration of 5 months. The samples are tested via broth culture against MRSA and E. Coli for over 24 hours at 37° C. inside an orbital shaker (200 RPM). The initial inoculum is 1 μmillion CFUs, which is depicted by the dotted line. A control polyurethane that was wiped with an antimicrobial CLOROX® disinfecting wipe (“cloroxed”) under similar conditions is used as a control. Error bars indicate one standard deviation. These results show that there was no detectable MRSA or E. coli in any of the antimicrobial coatings tested.

FIGS. 12A-12C show performance results of antimicrobial wound dressings under the broth culture method in an orbital shaker after 24 hours at 37° C. I represents an uncoated gauze. II-V represent antimicrobial wound dressings made in accordance with various aspects of the current technology (each including a matrix of BAYMEDIX® AR602 polyether polyol and BAYMEDIX® AP501 NCO-terminated prepolymer; II having 57 wt. % cinnamaldehyde and 3 wt. % α-terpineol, III having 30 wt. % cinnamaldehyde and 30 wt. % α-terpineol, IV having 60 wt. % α-terpineol, and V having 60 wt. % α-terpineol applied onto a surface of a thicker 12-ply gauze. VI—VII represent commercial antimicrobial dressing controls (SILVERLON® island dressings and SILVERLON® wound packing strips, respectively). VIII represents a control gauze with 0.5 g bacitracin. The dotted lines indicate an initial inoculum level. Error bars indicate one standard deviation. FIG. 12A shows results of dressings contacted with MRSA. FIG. 12B shows results of dressings contacted with E. coli (UTI189 strain). FIG. 12C shows results of dressings contacted with P. Aeruginosa (PA 27853 strain). FIG. 12D shows photographs of dressings I, II, and V.

FIG. 13 is a Fourier-transform infrared spectroscopy (FTIR) graph showing reaction kinetics of isocyanate and α-terpineol. The graph shows reduced absorbance of an —NCO peak at approximately 2260 cm⁻¹ over time, which indicates a reaction of the isocyanate with the α-terpineol in the presence of 0.01 wt. % DBTL. No isocyanate peaks are observed 5 days into the reaction.

FIG. 14 shows a TGA isotherm at 120° C. for an antimicrobial coating prepared in accordance with various aspects of the current technology (DESMOPHEN® polyurethane polymer matrix and 35 wt. % α-terpineol). About 31 wt. % of the α-terpineol is free and stabilized within the polyurethane matrix, while the remaining 4 wt. % α-terpineol is reacted covalently (bonded) within the polyurethane. The plot is normalized with the mass loss from control polyurethane.

FIG. 15 is a graph showing instant kill performance against E. coli. Using a modified version of ISO 22196, the surface of an antimicrobial coating prepared in accordance with various aspects of the current technology (DESMOPHEN® polyurethane polymer matrix and 35 wt. % α-terpineol) is tested against 10⁶ cells of E. Coli (UTI189 strain). DESMOPHEN® polyurethane and polystyrene are used as control surfaces. Error bars indicate one standard deviation.

FIGS. 16A-16C are time-elapsed fluorescence micrographs of E. coli on different surfaces. Cells are dyed (LIVE/DEAD® BACLIGHT™, ThermoFischer) and exposed to an antimicrobial coating prepared in accordance with various aspects of the current technology (DESMOPHEN® polyurethane polymer matrix and 35 wt. % α-terpineol), brass, and polyurethane, as shown in FIGS. 16A, 16B, and 16C, respectively. Under fluorescence microscopy, rapid cell death is observed within seconds for the case of DESMOPHEN® and 35% wt % α-terpineol. No live cells are observed at approximately 5 minutes. As for the naval brass and polyurethane controls, a majority of the cells remained alive over a period of one hour. The scale bar is 100 microns.

FIGS. 17A-17B are graphs showing instant kill performance against MRSA. Using a solid-solid contact plating method, the surface of an antimicrobial coating prepared in accordance with various aspects of the current technology (DESMOPHEN® polyurethane polymer matrix and 35 wt. % α-terpineol) is tested against 3000 cells and 10⁶ cells of MRSA, as shown in FIGS. 17A and 17B, respectively, to replicate minor and major contamination events. The transfer efficiency is 63.3% for DESMOPHEN® polyurethane, 35.3% for polystyrene, and 36.7% for DESMOPHEN® polyurethane and 35 wt % α-terpineol. Error bars indicate one standard deviation.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

The current technology provides antimicrobial compositions, methods of generating antimicrobial compositions, and methods of preparing antimicrobial surfaces. As used herein, the term “antimicrobial” refers to a composition that has at least one of an antibacterial, an antiviral, and an antifungal activity. The antimicrobial compositions of the current technology are not toxic to humans or domestic animals, but can be toxic to Acinetobacter baumanii, Actinomyces viscosus, Actinomyces spp., Aeromonas veronii bio-group sobria, Alternaria spp., Aspergillis fumigatus, A. flavus, A. niger, Bacillus cereus, B. subtillis, Bacteroides spp., Blastoschizomyces capitatus, Candida albicans, C. glabrata, C. parapsilosis, C. tropicalis, Cladosporium spp., Corynebacterium sp., Cryptococcus neoformans, Enterococcus faecalis, E. faecium (vancoymcin resistant), Epidermophyton flocossum, Escherichia coli, Fusarium spp., Fusobacterium nucleatum, H. influenzae, Klebsiella pneumoniae, Lactobacillus spp., Listeria monocytogenes, Malassezia furfur, M. sympodalis, M. catarrhalis, Micrococcus luteus, Microsporum canis, M. gypseum, Mycoplasma hominis, M. fermentans, M. pneumoniae, Penicillium spp., Peptostreptococcus anaerobis, Porphyromonas endodentalis, P. gigivalis, Prevotella spp., Prevotella intermedia, Propionibacterium acnes, Proteus mirabilis, Proteus vulgaris, Pseudomonas aeruginosa, R. sphaeroides, Rhodotorula rubra, Saccharomyces cerevisiae, Salmonella enterica subsp. enterica serotype typhimurium, Serratia marcescens, Staphylococcus aureus, S. aureus (methicillin resistant), S. epidermis, S. hominus, Streptococcus pyogenes, S. pneumonia, Trichophyton mentagrophytes, T. rubrum, T. tonsurans, Trichosporon spp., Veillonella spp., HSV-1 (herpes simplex virus) and HSV-2.

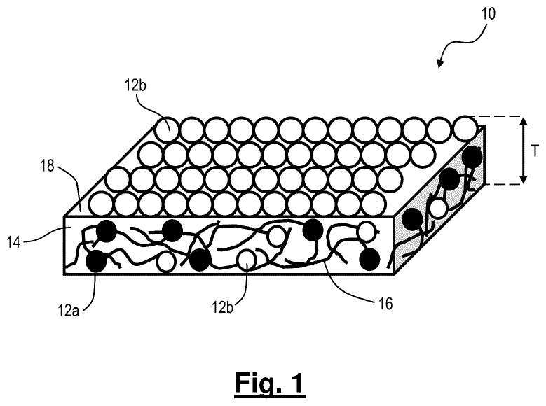

With reference to FIG. 1, the current technology provides an antimicrobial composition 10. The antimicrobial composition 10 comprises an oil-derived component 12a, an oil-derived antimicrobial component 12b, and a polymer matrix 14. The polymer matrix 14 is a matrix formed from a polymer 16. The oil-derived component 12a is covalently bonded to the polymer matrix 14. In this regard, the oil-derived component 12a comprises molecules having groups that react with reactive groups of monomers that polymerize to form the polymer 16, such that the oil-derived component 12a comprises molecules that are covalently bonded to a portion of monomers as the antimicrobial composition 10, including the polymer matrix 14, is formed. Therefore, the oil-derived component 12a comprises molecules that are covalently bonded to the polymers 16 that make up the polymer matrix 14. The oil-derived antimicrobial component 12b is not covalently bonded to the monomers that form the polymer matrix 14. Put another way, the oil-derived antimicrobial component 12b comprises molecules that are not covalently bonded to the polymers 16 that make up the polymer matrix 14. Rather, the oil-derived antimicrobial component 12b remains non-covalently associated, i.e., physically associated, with at least one of the oil-derived component 12a in the polymer matrix 14 and the polymer matrix 14. In some embodiments, the oil-derived antimicrobial component 12b remains non-covalently (physically) associated with at least the oil-derived component 12a in the polymer matrix 14. Whereas a first portion of the oil-derived antimicrobial component 12b can be non-covalently associated with the oil-derived components 12a in the polymer matrix 14, a second portion of the oil-derived antimicrobial component 12b can be located on a surface 18 of the polymer matrix 14 and remains non-covalently associated with the oil-derived component 12a. Without being bound by theory, it is most likely that the oil-derived antimicrobial component 12b remains non-covalently associated with at least one of the oil-derived component 12a and the polymer matrix 14 by van der Waals forces.

As used herein, components that are “oil-derived” are components and/or molecules that are donated from or isolated from an oil, or that are synthesized as copies of components and/or molecules that are found in an oil, wherein the oil is a plant or seed extract. The plant or seed extract can have antimicrobial activity provided by antimicrobial molecules, or the plant or seed extract may not have antimicrobial activity. Regarding the oil-derived component 12a, in some embodiments it is donated or isolated from a non-antimicrobial plant or seed extract and comprises molecules that do not have antimicrobial activity. In such embodiments, the oil-derived component 12a does not have antimicrobial activity. In other embodiments, the oil-derived component 12a is donated or isolated from an antimicrobial plant or seed extract, or is at least one synthesized molecule that is naturally found in a plant or seed extract, and comprises molecules that have antimicrobial activity. In these embodiments, the oil-derived component 12a has antimicrobial activity. In all embodiments, the oil-derived antimicrobial component 12b is donated or isolated from an antimicrobial plant or seed extract, or is at least one synthesized molecule that is naturally found in a plant or seed extract, and comprises molecules having antimicrobial activity. As such, the oil-derived antimicrobial component 12b can be an antimicrobial oil (i.e., an oil comprising molecules that exhibit antimicrobial activity), molecules isolated or donated from an antimicrobial oil and that have antimicrobial activity, or at last one synthesized antimicrobial molecule that is naturally found in an antimicrobial oil, wherein the molecules exhibit antimicrobial activity. Therefore, in some embodiments of the current technology, the oil-derived component 12a and the oil-derived antimicrobial component 12b comprise the same oil-derived antimicrobial molecules. In other embodiments of the current technology, the oil-derived component 12a comprises molecules (antimicrobial or non-antimicrobial) from an antimicrobial oil and the oil-derived antimicrobial component 12b comprises the antimicrobial oil non-covalently associated within the polymer matrix 14.

The oil-derived component 12a and the oil-derived antimicrobial component 12b have a combined concentration in the antimicrobial composition 10 of greater than or equal to about 1 wt. % to less than or equal to about 95 wt. %, greater than or equal to about 5 wt. % to less than or equal to about 80 wt. %, greater than or equal to about 10 wt. % to less than or equal to about 75 wt. %, or greater than or equal to about 15 wt. % to less than or equal to about 70 wt. %. Moreover, the oil-derived component 12a and the oil-derived antimicrobial component 12b are present in an oil-derived component:oil-derived antimicrobial component ratio of from about 1:100 to about 100:1, from about 1:10 to about 10:1, from about 1:4 to about 4:1, from about 1:3 to about 3:1, or from about 1:2 to about 2:1. The oil-derived component:oil-derived antimicrobial component ratio depends upon both the amount of reactive oil components that can form covalent bonds with the polymer 16 and the ability of the monomers that form the polymer 16 to form covalent bonds with the oil-derived component 12a.

The oil-derived antimicrobial component 12b of the antimicrobial composition 10 provides all or most of the antimicrobial activity of the antimicrobial composition 10. Therefore, at least a portion of the oil-derived antimicrobial component 12b has antimicrobial activity. Oil-derived molecules that exhibit antimicrobial activity (i.e., antimicrobial molecules) include alkaloids, glycosides, terpenes (including hemiterpenes, monoterpenes, sesquiterpenes, diterpenes, sesterterpenes, triterpenes, sesquarterpenes, tetraterpenes, polyterpenes, and norisoprernoids, and including α-terpinene, β-terpinene, γ-terpinene, δ-terpinene cymenes and linalool), terpenoids/isoprenoids (including hemiterpenoids, monoterpenoids, sesquiterpenoids, diterpenoids, sesterterpenoids, triterpenoids, tetraterpenoids, and polyterpenoids, and including the monoterpenoids carvacrol, thymol, menthol, carvone, limonene, eucalyptol, camphor and borneol, and the terpenoids α-terpineol, β-terpineol, γ-terpineol, terpinen-4-ol, citral, citronellal, citronellol and geraniol), saponins, steroids, flavonoids (including anthoxanthins, flavanones, flavanonols, flavans, and anthocyanidins), isoflavonoids (including isoflavones, isoflavanes, isoflavandios, isoflavenes, coumestans, and pterocarpans), phenolics/polyphenols (including flavones, quinones, and tannins), phenylpropanoids (such as cinnamaldehyde), phenylpropenes (such as eugenol, chavicol, safrole and estragole) coumarins, curcuminoids (such as curcumin, dimethoxycurcumin, and bisdemethoxycurcumin) and combinations thereof, as non-limiting examples.

In various embodiments, at least the oil-derived antimicrobial component 12b, and, optionally, the oil-derived component 12a, is isolated or donated from a natural oil, such as an oil extracted from basil (Ocimum basilicum), bergamot (Citrus aurantium bergamia), black pepper (Piper nigrum), Brazil's spearmint (Mentha arvensis), cardamom (Elettaria cardamomum), cedar (Cedrus atlantica), cinnamon (Cinnamomum cassia), citron (Citrus medica), clary sage (Salvia sclarea), clove (Syzygium aromaticum), copaiba (Copaifera officinalis), coriander (Coriandrum sativum), cypress (Cupressus sempervirens), eucalyptus (Eucalyptus globulus), fennel (Foeniculum vulgare), geranium (Pelargonium graveolens), ginger (Zingiber officinalis), lavender (Lavandula angustifolia), lemongrass (Cymbopogon schoenanthus), mandarin (Citrus reticulata), marjoram (Origanum majorana), nutmeg (Myristica fragans), orange (Citrus aurantium dulcis), oregano (Origanum vulgare), palmarosa (Cymbopogon martinii), patchouli (Pogostemon patchouli), peppermint (Mentha piperita), perilla (Perilla frutescens), pine (Pinus sylvestris), rosemary (Rosmarinus officinallis), Tahiti lime (Citrus limonum), tea tree (Melaleuca alternifolia), thyme (Thymus vulgaris), vetiver (Vetiveria zizanioides), ylang ylang (Cananga odorata), Achillea clavennae, Achillea fragrantissima, Achillea, Achillea ligustica, Artemisia absinthium, Artemisia biennis, Artemisia cana, Artemisia dracunculus, Artemisia longifolia, Artemisia frigida, Cinnamomum zeylancium, Copaifera officinalis, Cuminum cyminum, Cymbopogon citratus, Cymbopogon nardus, Cyperus longus, Daucus littoralis, Dracocephalum foetidum, Eremanthus erythropapps, Eugenia caryophyllata, Euphrasia rostkoviana, Fortunella margarita, Juniperus phoenicea, Laurus nobilis, Juniperus excelsa, Lippia sidoides, Mentha pulegium, Mentha suaveolens, Momordica charantia, Myrtus communis, Nigella sativa, Ocimum gratissimum, Ocimum kilimandscharicum, Origanum vulgare, Ocimum basilicum, Petroselinum sativum, Piper nigrum, Pimpinella anisum, Plectranthus barbatus, P. amboinicus, Plectranthus neochilus, Pogostemon cablin, Rosmarinus officinalis, Satureja hortensis, Salvia officinalis, Salvia lavandulifolia, Satureja cuneifolia, Struchium sparganophora, Syzygium cumini, Trachyspermum ammi, Thymus zygis, Thymus mastichina, Thymus kotschyanus, Thuja sp. (Thuja plicata, Thuja occidentalis), Verbena officinalis, Warionia saharae, fractions thereof, components thereof, molecules thereof, or combinations thereof, as non-limiting examples. It is understood that although specific species of each plant are provided, other species of each plant may provide antimicrobial oil extracts as well. In some embodiments, at least one component, fraction or molecule of the foregoing oils is the oil-derived component 12a, the oil-derived antimicrobial component 12b, or both the oil-derived component 12a and the oil-derived antimicrobial component 12b combined with the polymer matrix 14.

The polymer 16 defining the polymer matrix 14 can be any polymer that has reactive groups capable of forming covalent bonds with the oil-derived component 12a. Non-limiting examples of suitable polymers 16 include polyurethane (comprising a polyisocyanate or dicarboxylic acid and a polyol), polyethers, polycarbonates, polyaspartics, polyesters (including polyethylene terephthalate (PET)), polyolefin, acrylates, poly(acrylic acid) (PAA), poly(methyl acrylate) (PMA), poly(methyl methacrylate) (PMMA), acrylonitrile butadiene styrene (ABS), polyamides (including polycaprolactam (nylon)), polylactic acid (PLA), polybenzimidazole, polycarbonate, polyether sulfone (PES), polyetherether ketone (PEEK), polyetherimide (PEI), polyethylene (PE; including ultra-high molecular weight polyethylene (UHMWPE), medium-density polyethylene (MDPE), low-density polyethylene (LDPE), and cross-linked polyethylene (PEX)), polyphenylene oxide (PPO), polyphenylene sulfide (PPS), polypropylene (PP), polystyrene (PS), polyvinyl chloride (PVC), polytetrafluoroethylene (PTFE), polyimides, vinyl esters, epoxy, polydimethylsiloxane, polyurethane (PU), perfluoropolyether (PFPE), polymethylhydrosiloxane (PMHS), polymethylphenylsiloxane (PMPS), copolymers of isocyanate functionalized polydimethylsiloxane (PDMS) and fluorinated polyurethane (FPU), copolymers of isocyanate functionalized polydimethylsiloxane (PDMS) and polyurethane (PU), acrylates, methacrylates, soybean oil acrylate, polystyrene, natural rubber, vulcanized rubber, synthetic rubber, butyl rubber, latex rubber, polychloroprene, acrylonitrile butadiene rubber, styrene butadiene rubber, elastomers made from ethylene propylene diene monomer (EPDM), epichlorohydrin-based rubber, poly(lactic-co-glycolic acid) (PLGA), epoxy, organogels, hydrogels, other elastomers, copolymers thereof, and combinations thereof, as non-limiting examples.

The antimicrobial composition 10 has a thickness T of greater than or equal to about 1 μm to less than or equal to about 100 mm, greater than or equal to about 10 μm to less than or equal to about 10 mm, greater than or equal to about 100 μm to less than or equal to about 8 mm, greater than or equal to about 400 μm to less than or equal to about 5 mm, or greater than or equal to about 500 μm to less than or equal to about 3 mm. However, it is understood that the thickness of the antimicrobial composition 10 is only limited by the size of a border or mold used to contain the antimicrobial composition 10 in a particular location. Therefore, the antimicrobial composition 10 is provided as a solid film or a solid layer, or as an abstract shape defined by a die or mold. Put another way, the antimicrobial composition 10 can be an antimicrobial film or layer applied onto a preexisting surface, or the antimicrobial composition 10 can define an abstract shape that has antimicrobial properties. The antimicrobial composition 10 can be rigid or elastomeric and transparent or opaque, based on the polymer 16 and the thickness T of the antimicrobial composition 10. In various embodiments, the antimicrobial composition 10 is visibly transparent with a visible light transmission of greater than or equal to about 50%, greater than or equal to about 60%, greater than or equal to about 70%, or greater than or equal to about 80%. Moreover, the antimicrobial composition 10 (or its surface 18) can be textured or microtextured, i.e., having lines, grooves, or contours that are visible to the human eye or invisible to the human eye. Some textures or microtextures can further prevent microbial adhesion to the antimicrobial composition 10. The textures or microtextures can be a result of processing techniques used to apply the antimicrobial composition 10 onto the polymer matrix 14, such as spraying, brushing, and dip coating, as non-limiting examples.

The antimicrobial composition 10 has instant antimicrobial activity. As used herein, the term “instant antimicrobial activity” means that the antimicrobial composition 10 kills at least about 50%, at least about 60%, at least about 75%, at least about 80%, at least about 90%, at least about 95%, or at least about 99% of the microbes that come in contact with the antimicrobial composition 10 within a time period of less than or equal to about 45 minutes, less than or equal to about 30 minutes, less than or equal to about 20 minutes, less than or equal to about 15 minutes, less than or equal to about 12 minutes, less than or equal to about 11 minutes, less than or equal to about 10 minutes, less than or equal to about 8 minutes, less than or equal to about 6 minutes, or less than or equal to about 3 minutes. Therefore, the term “instant antimicrobial activity” refers to a potency.

The antimicrobial composition 10 also has a persistent antimicrobial activity. As used herein, the term “persistent antimicrobial activity” means that the antimicrobial composition 10 retains antimicrobial activity, i.e., the antimicrobial activity persists for a time period of greater than or equal to about 2 days, greater than or equal to about 1 week, greater than or equal to about 2 weeks, greater than or equal to about 1 month, greater than or equal to about 2 months, greater than or equal to about 3 months, greater than or equal to about 4 months, greater than or equal to about 5 months, or greater than or equal to about 6 months. Therefore, the term “persistent antimicrobial activity” refers to an effective duration. Moreover, when the antimicrobial composition 10 loses its antimicrobial activity, the antimicrobial composition 10 can be rejuvenated, i.e., the antimicrobial activity can be restored, by applying a water-based solution or emulsion comprising the antimicrobial oil onto the antimicrobial composition 10. The applying can be performed by spraying, wiping, pouring, or by any other method that covers the antimicrobial composition with the solution or emulsion. Applying the water-based solution or emulsion comprising the antimicrobial oil onto the antimicrobial composition 10 provides new oil-derived antimicrobial components 12b of the oil to become physically associated with at least one of the oil-derived components 12a, which remain in the polymer matrix 14, and the polymer matrix 14 itself.

By controlling the fraction of the oil-derived component 12a covalently bonded within the polymer matrix 14, the immediate and persistent natures of action of the antimicrobial composition 10 can be controlled. For example, an antimicrobial composition 10 with a high fraction of the free oil-derived antimicrobial component 12b and a relatively low fraction of the covalently bonded oil-derived component 12a will generally require a shorter time to kill microbes present on the antimicrobial composition 10 (i.e., be more immediate), but generally will not persist over the long term, as the antimicrobial oil will evaporate in a shorter time period (i.e., be less persistent). Conversely an antimicrobial composition 10 with a high fraction of the covalently bonded oil-derived component 12a and a relatively lower fraction of the free oil-derived antimicrobial component 12b will generally demonstrate the opposite behavior, i.e., be less immediate, but more persistent. Accordingly, the oil-derived component:oil-derived antimicrobial component ratio (as defined above) can be adjusted for different applications. For example, an antimicrobial coating for a wound dressing may need to provide a very fast performance (i.e., be immediate), but may only require an active time period of a few days (i.e., be not very persistent). Other coating, for example for a cell phone cover, may require more persistent action (over several months), but may be suitable having a microbial kill time of approximately 30 minutes. Controlling the fraction of the oil-derived component 12a and the oil-derived antimicrobial component 12b, i.e., the oil-derived component:oil-derived antimicrobial component ratio, can be performed by selecting an antimicrobial oil having a low or high concentration of reactive groups and by selecting a polymer that is either highly reactive or not very reactive. As provided above, the oil-derived component:oil-derived antimicrobial component ratio depends upon both the amount of reactive oil components that can form covalent cross-links with the polymer 16 and the ability of the monomers that form the polymer 16 to form covalent bonds with the oil-derived component 12a. In one embodiment, the antimicrobial composition 10 comprises greater than or equal to about 33% to less than or equal to about 66% of the oil-derived component 12a, with the remainder being the oil-derived antimicrobial component 12b, and the antimicrobial composition 10 retains antimicrobial activity for a time period of greater than or equal to about 3 months.

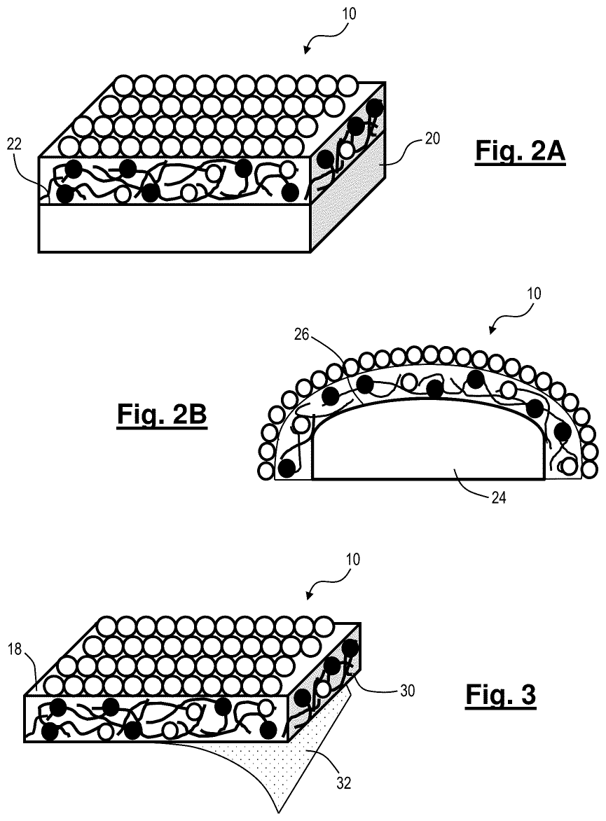

FIG. 2A also shows the same antimicrobial composition 10 that is shown in FIG. 1. However, in FIG. 2A the antimicrobial composition 10 is disposed on a substrate 20 having a flat or planar surface 22. FIG. 2B also shows the antimicrobial composition 10 that is shown in FIG. 1. However, in FIG. 2B the antimicrobial composition 10 is disposed on a substrate 24 having a curved or irregularly shaped surface 26. In various embodiments, the surfaces 22, 26 are high-touch surfaces. As used herein, “high-touch” surfaces are surfaces that come into human contact, such as surfaces of a child care facility, a hospital, a retirement home, a bathroom, a kitchen, or a vehicle (including automobiles, motorcycles, boats, recreational vehicles, tanks, airplanes, and bicycles, as non-limiting examples). The surface can be composed of any material, such as a metal, a polymer, a glass, a marble, a plastic, a quartz, or a steel, as non-limiting examples. Non-limiting examples of high-touch surfaces include surfaces of a counter, a toilet, a sink, flooring, tiles, a dashboard, a handhold, a handle, a door handle, a door knob, a handrail, a cup holder, a touch screen, a tray, a tray table, furniture, paint, a table, a chair, a seat, a fabric, a gear shifter, and a steering wheel. In other embodiments, the surfaces 22, 26 are surfaces that are configured to come into contact with an internal tissue, such as skin, blood, bone, or an organ tissue, and may be a surface 22, 26 of a medical implant or a wound dressing, as non-limiting examples. In any embodiment, because the antimicrobial composition 10 can be formed directly on the surfaces 22, 26, the topology of the surfaces 22, 26 are non-limiting. Additionally, because the antimicrobial composition 10 can be elastomeric, the surfaces 22, 26 can be flexible or pliable. Accordingly, the current technology further provides a high-touch surface comprising the antimicrobial composition 10.

In various embodiments, the antimicrobial composition 10 is formed or generated directly on a substrate. In other embodiments, the antimicrobial composition 10 is applied to a surface after the antimicrobial composition 10 is formed or generated. For example, FIG. 3 shows the same antimicrobial composition 10 described with reference to FIGS. 1, 2A, and 2B. However, the antimicrobial composition 10 of FIG. 3 comprises the surface 18 and an opposing second surface 30. The second surface 30 comprises an adhesive layer that is covered by a non-adhesive sheet or material 32. The antimicrobial composition 10 is applied to surface by first removing the non-adhesive sheet or material 32 to expose the second surface 30 having the adhesive layer, and then disposing the non-adhesive sheet or material 32 of the second surface 30 onto a substrate, similar to removing a sticker from a backing and applying the sticker to surface. In another embodiment, the antimicrobial composition 10 of FIG. 1 is applied to a substrate by way of an adhesive or glue.

As mentioned above, the antimicrobial composition 10 of FIG. 1 can be generated such that it defines a shape. In some embodiments, the antimicrobial composition 10 is in the form of a medical implant or prosthesis. The medical prosthesis can be an artificial joint, such as a knee, a hip, an elbow, or a shoulder. The medical implant can be a medical device that is implanted anywhere within a body, such as in a foot, a leg, a hip, a spine, a hand, an arm, a shoulder, a chest, or a skull. In other embodiments, the medical implant or prosthesis is obtained commercially and coated with the antimicrobial composition 10 by a method described herein. Accordingly, the current technology also provides medical implants and prostheses having a surface comprising the antimicrobial composition. The medical implants and prostheses are either composed of the antimicrobial composition 10 or are coated by the antimicrobial composition 10.

As described above, the antimicrobial composition 10 includes the covalently bonded oil-derived component 12a and the free (non-covalently bonded, i.e., physically associated) oil-derived antimicrobial component 12b. However, in some embodiments, the oil-derived component 12a is a component of an oil that does not have antimicrobial properties, but which is capable of becoming physically associated with, and stabilizing, the free oil-derived antimicrobial component 12b, which is derived from an antimicrobial oil. In such embodiments, the free oil-derived antimicrobial component 12b remains non-covalently associated, i.e., physically associated, with at least one of the oil-derived component 12a (which is not antimicrobial) in the polymer matrix 14 and the polymer matrix 14 itself.

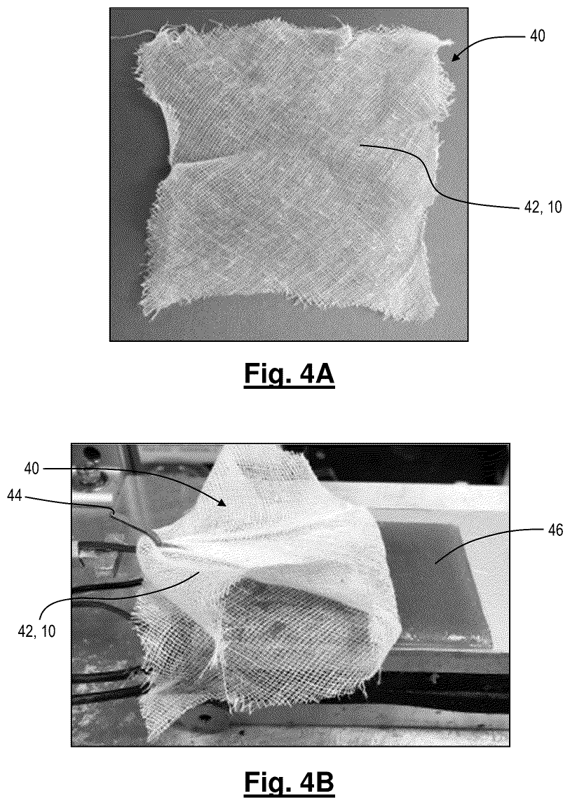

Also, as mentioned above, in various embodiments, the antimicrobial composition 10 is disposed within a wound dressing, such that the wound dressing comprises an antimicrobial surface. As an example, FIG. 4A shows a wound dressing 40 having a surface 42 comprising the antimicrobial composition 10 described with reference to FIG. 1. Although the wound dressing 40 shown in FIG. 4A is a gauze, the wound dressing can be any dressing used in the art, such as a gauze, a bandage, or a cloth, as non-limiting examples. The wound dressing 40 can be composed of a hydrocolloid, a hydrogel, an alginate, a collagen, a foam, a transparent material, or a cloth, as non-limiting examples of wound dressing materials. The antimicrobial composition 10 is elastomeric and embedded with the wound dressing 40. Therefore, the wound dressing 40 having an antimicrobial surface 42, 10 can be used to kill microbes in a wound or to inhibit microbe growth within a wound. The wound can be any wound that requires medical attention, such as a cut, a gash, a scrape, a surgical scar, a surgical wound, or a diabetic ulcer, as non-limiting examples.

Furthermore, the wound dressing 40 is less adhesive to a wound than a second dressing comprising the same dressing material, but without the antimicrobial composition. As shown in FIG. 4B, an adhesion test can be conducted on the wound dressing 40 comprising the antimicrobial surface 42, 10. A mechanical arm 44 attached to a sensor (not shown) is used to pull the wound dressing 40 off of a material 46 that mimics a wound. A second control wound dressing comprising the same material as the wound dressing 40, but without the antimicrobial composition 10, is also tested in the same manner. The wound dressing 40 comprising the antimicrobial surface 42, 10 is removed with less adhesive force than the second control wound dressing.

The current technology also provides a method for forming or generating an antimicrobial composition. The method comprises combining an antimicrobial oil or oil-derived antimicrobial molecules (or both) with an uncured polymer precursor solution to form a mixture. The antimicrobial oil can be any natural plant or seed oil having antimicrobial compounds described herein. The uncured polymer precursor solution comprises at least one monomer and, optionally, an initiator. The at least one monomer is one or more monomers that are needed in order to form a desired polymer. Exemplary polymers are described above. The optional initiator is a chemical initiator, a catalyst, a cross-linking agent, a thermal-induced initiator, a photo-induced initiator, a base, or an acid, as non-limiting examples, that initiates, catalyzes, or speeds up a polymerization chain reaction (i.e., addition reaction) by, for example, inducing the formation of reactive free radicals. In various embodiments, the mixture comprises the antimicrobial oil or oil-derived antimicrobial molecules at a concentration of greater than or equal to about 1 wt. % to less than or equal to about 95 wt. %, greater than or equal to about 10 wt. % to less than or equal to about 75 wt. %, or greater than or equal to about 15 wt. % to less than or equal to about 60 wt. %.

The method then comprises curing the mixture to generate the antimicrobial composition. The antimicrobial composition, as described herein, comprises a polymer matrix formed from the uncured polymer precursor solution, an oil-derived component covalently bonded to the polymer matrix, and an oil-derived antimicrobial component non-covalently associated with at least one of the polymer matrix and the oil-derived component. The oil-derived component and the oil-derived antimicrobial component are provided from the antimicrobial oil or the oil-derived antimicrobial molecules. The oil-derived component and the oil-derived antimicrobial component are present in an oil-derived component:oil-derived antimicrobial component ratio of from about 1:100 to about 100:1, from about 1:10 to about 10:1, from about 1:4 to about 4:1, from about 1:3 to about 3:1, or from about 1:2 to about 2:1. The oil-derived component:oil-derived antimicrobial component ratio depends upon both the amount of reactive oil components that can form covalent cross-links with the polymer and the ability of the monomers that form the polymer to form covalent cross-links with oil components.

The curing includes cross-linking the oil-derived component of the antimicrobial oil or the oil-derived antimicrobial molecules to a portion of the at least one monomer and polymerizing a remaining portion of the at least one monomer to form the polymer matrix with the oil-derived component of the antimicrobial oil or the oil-derived antimicrobial molecules covalently bonded thereto. The curing is performed under conditions that are appropriate for polymerizing the at least one monomer. For example, whereas some reactions occur at room temperature without an initiator, other reactions occur at a temperature greater than room temperature without an initiator. Further, some reactions require an initiator and an activator that activates the initiator. Non-limiting examples of activators include heat, light (ultraviolet, visible, or infrared), electricity, and chemicals.

In some embodiments, the method further comprises applying the mixture to a surface of a substrate before the curing. The substrate can be any substrate described herein, such as an object with a high-touch surface or a medical implant or prosthesis. Therefore, an antimicrobial film, layer, or coating is disposed onto a substrate after performing the method. The substrate can also be a temporary substrate. Accordingly, in various embodiments, the method yet further comprises, after the curing, removing the antimicrobial composition from the substrate to isolate an antimicrobial film comprising the antimicrobial composition. The antimicrobial film can be disposed onto another substrate, such as a substrate having a high-touch surface or a medical implant or prosthesis, with, for example, an adhesive or glue. Alternatively, the method can further comprise disposing an adhesive onto a surface of the antimicrobial film and covering the adhesive with a non-adhesive sheet or material. This antimicrobial film can be applied onto a desired surface by removing the non-adhesive sheet or material to expose the adhesive on the surface of the antimicrobial film and disposing the adhesive surface onto the desired surface.

In other embodiments, the method further comprises disposing a wound dressing into the mixture and performing the curing while the wound dressing is disposed in the mixture. After the curing, an antimicrobial wound dressing comprising the antimicrobial composition, such as any antimicrobial wound dressing described herein, is formed.

In yet other embodiments, the method further comprises transferring the mixture into a mold or die and performing the curing with the mixture in the mold or die. After the curing, an object having a predetermined shape is formed, wherein the object has an antimicrobial surface. The object can be any object having a high-touch surface or a medical implant or prosthesis that can be made in a mold or die.

As mentioned above, in some embodiments, the covalently bonded oil-derived component is not antimicrobial. In such embodiments, the method includes combining a non-antimicrobial oil or oil-derived molecules that do not have antimicrobial activity with an uncured polymer precursor solution to form a mixture and curing the mixture to form a hardened composition comprising non-antimicrobial oil-derived components covalently bonded to a polymer matrix. An antimicrobial oil or oil-derived antimicrobial molecules are then contacted with the hardened composition by dunking, spraying, or brushing, as non-limiting examples. Antimicrobial components of the antimicrobial oil or oil-derived antimicrobial molecules become non-covalently associated, i.e., physically associated, with at least one of the covalently bonded oil-derived component (which is not antimicrobial) in the polymer matrix and the polymer matrix itself.

The current technology also provides a method of preparing an antimicrobial surface. The method comprises applying a mixture to a surface, the mixture comprising an antimicrobial oil or oil-derived antimicrobial molecules, an uncured polymer precursor solution, and, optionally, an initiator. The antimicrobial oil or oil-derived antimicrobial molecules and the uncured polymer precursor solution can be any antimicrobial oil or oil-derived antimicrobial molecules and uncured polymer precursor solution described herein. The method also comprises incubating the mixture on the surface until the mixture cures and forms an antimicrobial composition, such as a film on the surface, for example. The incubating can be for a time period of greater than or equal to about 5 minutes to less than or equal to about 1 week, greater than or equal to about 10 minutes to less than or equal to about 3 days, or greater than or equal to about 1 hour to less than or equal to about 1 day. As described above, the antimicrobial film comprises the antimicrobial oil or oil-derived antimicrobial molecules and a polymer matrix, wherein an oil-based component is covalently bonded to the polymer matrix and an oil-derived antimicrobial component is non-covalently associated with the oil-derived component and/or the polymer matrix. The surface can be any surface described herein, including a wound dressing surface, a high-touch surface of an object, and a surface of a medical implant or prosthesis.

In some embodiments, the covalently bonded oil-derived component is not antimicrobial. In such embodiments, the method includes combining a non-antimicrobial oil or oil-derived non-antimicrobial molecules with an uncured polymer precursor solution to form a mixture, applying the mixture to the surface, and curing the mixture to form a hardened composition comprising non-antimicrobial components covalently bonded to a polymer matrix. An antimicrobial oil or oil-derived antimicrobial molecules is then contacted with or coated onto the hardened composition by dunking, spraying, or brushing, as non-limiting examples. Antimicrobial components of the antimicrobial oil or oil-derived antimicrobial molecules become non-covalently associated, i.e., physically associated, with at least one of the covalently bonded oil-derived components (which are not antimicrobial) in the polymer matrix and the polymer matrix itself.

In some embodiments, the antimicrobial composition mixture is prepared from a kit comprising at least one uncured monomer, the antimicrobial oil or the oil-derived antimicrobial molecules, and, optionally, at least one of an initiator and an activator. The kit is also provided by the current technology.

The current technology yet further provides a method of rejuvenating an antimicrobial surface prepared by the above method of preparing an antimicrobial surface. The method comprises applying a water-based solution, an emulsion comprising fresh antimicrobial oil, or a fresh antimicrobial oil to the antimicrobial surface and incubating the antimicrobial surface until the antimicrobial oil becomes physically associated with the antimicrobial film. As used herein, the term “fresh” refers to a composition or oil that is newly made or acquired.

The current technology also provides a method of making an antimicrobial object.

The method comprises transferring a mixture to a mold or die, the mixture comprising an antimicrobial oil or oil-derived antimicrobial molecules, an uncured polymer precursor solution, and, optionally, an initiator. The antimicrobial oil or oil-derived antimicrobial molecules and the uncured polymer precursor solution can be any antimicrobial oil, oil-derived antimicrobial molecules, and uncured polymer precursor solution described herein. The method also comprises incubating the mixture in the mold or die until the mixture cures and forms an antimicrobial object. The incubating can be for a time period of greater than or equal to about 5 minutes to less than or equal to about 1 week, greater than or equal to about 10 minutes to less than or equal to about 3 days, or greater than or equal to about 1 hour to less than or equal to about 1 day. The antimicrobial object has an antimicrobial surface. The antimicrobial object can be any object described above in relation to this method.

In some embodiments, the mixture comprises combining a non-antimicrobial oil or oil-derived non-antimicrobial molecules, with the uncured polymer precursor solution, and, optionally, an initiator to form a mixture, and incubating the mixture in the mold or die until the mixture cures and forms a hardened object. An antimicrobial oil or oil-derived antimicrobial molecules is then contacted with or coated onto the hardened object by dunking, spraying, or brushing, as non-limiting examples. Antimicrobial components of the antimicrobial oil or the oil-derived antimicrobial molecules become non-covalently associated, i.e., physically associated, with at least one of the covalently bonded oil-derived component (which is not antimicrobial) in the polymer matrix and the polymer matrix itself.

In some embodiments, the mixture is prepared from a kit as provided above.

Embodiments of the present technology are further illustrated through the following non-limiting example.

### Example

Antifouling and antibacterial surfaces are of interest due to a plethora of potential applications. Many natural oils, including eucalyptus oil, tea tree oil, patchouli oil, geranium oil, and lavender oil, among others, possess antimicrobial properties. However, these oils are typically volatile, and depending on the environment, can evaporate from a surface within a few minutes to several hours. Here, long-lasting (greater than 3 months) antimicrobial surfaces are produced by partially cross-linking different natural oils within a cross-linkable polymer matrix, such as a polyurethane. The cross-linkable polymer matrix is chosen such that it can chemically react with at least some components of the natural oil. The cross-linked components then serve to stabilize the remaining portion of the oil, referred to herein as “free oil,” within the polymer matrix for extended periods of time. This approach can be used for different natural oils; however, there is an optimal amount of cross-linking required to produce long-lasting antimicrobial surfaces, as if the cross-linking is too small, the oil is not fully stabilized and the surface will be unable to maintain its antimicrobial properties over the long term, and if too much oil is cross-linked, the surface is no longer antimicrobial, due to very small amounts of free oil. The “optimal amount” depends on how immediate and persistent the antimicrobial composition is desired to be. For example, an antimicrobial composition comprising from about 33% to about 66% of cross-linked oil, with the remainder of the oil being free (i.e., physically associated) is generally considered “long-lasting.” One measure of the stabilization of the free oil present in the polymer matrix is a change in an evaporation rate at room temperature of the free oil in the polymer matrix with the cross-linked oil relative to an evaporation rate of the free oil in a polymer matrix without any cross-linked oil. A long-lasting antimicrobial surface would require between a 1-99% reduction in the evaporation rate of the free oil.

Here, an antimicrobial essential oil is chemically reacted into the polymer as the diisocyanate and polyol simultaneously react to form a polyurethane. Specifically, tea tree oil is focused on. Tea tree oil is a well-known, natural, antibacterial oil that is used for the treatment of different infections. The oil is non-toxic, has anti-inflammatory properties, and is approved as an active agent for use within wound care by the FDA. Another natural antimicrobial oil, eucalyptus oil, can similarly be cross-linked within a polyurethane matrix, using the same polyol-isocyanate bond. The result is an antifouling polyurethane with a partial amount of “free” essential oil within the polymer network stabilized by the rest of the cross-linked essential oil. The tea tree oil containing polyurethane is highly abrasion resistant and is capable of reducing bacteria adhesion by at least 99%, even when left exposed to air for 12 weeks.

### Methods

### Surface Fabrication.

Polystyrene (PS) surfaces: Surfaces are fabricated using sterile PS petri dishes obtained from Fischer Scientific. The surfaces are cleaned and then exposed to UV light for 30 minutes to guarantee sterility.

Polyurethane (PU) surfaces: DESMOPHEN® 670 BA (polyol) and DESMODUR® N3800 (diisocyanate) are purchased from Covestro and mixed at a weight ratio of 0.5363:0.4637, respectively. Essential oils, tea tree (TTO) and eucalyptus oil (purchased from Jedwards International, Inc.), and essential oil components (Sigma) are added to the uncured polyurethane mixture by weight percent, where 30% oil equals 30% of the total polyurethane plus oil weight. The solutions are then drop casted onto a glass slide, allowed to cure in a chemical fume hood for at least 4 days, and then are exposed to UV light for 30 minutes to guarantee sterility. Typical coating thickness is about 1.5-2 mm.

Polydimethylsiloxane (PDMS) surfaces: MOLD MAX STROKE® (Smooth-On Inc.) is mixed in a 10:1 base:cross-linker ratio, following the manufacturer's instructions. 10 g of total material is taken and the mixture is vortexed until homogeneous. To make an antimicrobial sample, 30 wt. % tea tree oil is added and vortexed. The mixture is then cast over a glass slide, allowed to cure in a chemical fume hood for at least 4 days, and then is exposed to UV light for 30 minutes to guarantee sterility. Typical coating thickness is about 1.5-2 mm.

Epoxy surfaces: 100 parts of EPOXACAST® 650 (Smooth-On Inc.) is mixed thoroughly with 12 parts of 101 Hardener, following the manufacturer's instructions. 10 g of total material is taken and the mixture is stirred until homogeneous. To make an antimicrobial sample, 30 wt. % tea tree oil is added and vortexed. The mixture is then cast over a glass slide, allowed to cure in a chemical fume hood for at least 24 hours, and then is exposed to UV light for 30 minutes to guarantee sterility. Typical coating thickness is about 1.5-2 mm.

### Wound Dressing Fabrication.

Stoichiometric quantities of BAYMEDIX® AR602 polyether polyol are mixed with BAYMEDIX® AP501 NCO-terminated prepolymer (based on hexamethylene diisocyanate) in a vortexer. Oil components are added in the remaining parts. 0.01-0.10 wt. % bismuth neodecanoate is added to the mixture before the uncoated gauze (from Curity) is immersed into the vat. Excess resin is strained out and the resulting coated gauze is cured for at least 24 hours. Table 1 details the compositions (in wt. %).

TABLE 1
Components of wound dressing compositions.

| | | | | Cinnamal- |
|---|---|---|---|---|
| | AP501 | AR602 | α-terpineol | dehyde |
| | (isocyanate) | (polyol) | (α-t) | (CMA) |
| BM | 11.8 | 88.2 | — | — |
| (BAYMEDIX ®) | | | | |
| BM + 60% α-t | 9.2 | 30.8 | 60 | — |
| BM + 30% α-t + 30% | 9.2 | 30.8 | 30 | 30 |
| CMA | | | | |
| BM + 57% α-t + 3% | 9.2 | 30.8 | 57 | 3 |
| CMA | | | | |

SILVERLON® island dressings and SILVERLON® wound packing strips are purchased from Amazon and cut into desired dimensions of 2 cm×1 cm. Bacitracin petroleum gel is acquired from Dynarex and spread along the walls of the aliquot, which contains the media.

### Surface Characterization.

Contact angle measurements: Dynamic contact angles are measured with water on the polyurethane surfaces with a Ramé-Hart 200-F1 goniometer, using the sessile drop technique.

Thermogravimetric analysis (TGA): Weight loss measurements are conducted on the Q5000IR by TA Instruments. The weight loss of the samples is observed under nitrogen atmosphere and an isothermal temperature of 200° C. for 200 minutes after a ramp of 10° C./minute. Weight loss percentage is recorded at 100 minutes for each sample.

Gas Chromatography—Mass Spectroscopy (GC-MS): Oil sample composition is determined using the Shimadzu QP-2010 GCMS consisting of a Supelco SLB®−5 ms Capillary GC Column (L×I.D. 30 m×0.25 mm, df 0.25 μm). The sample is injected at a temperature of 200° C. using split mode (split ratio=100), and the mass spectrometer is operated in scan mode with a mass range (m/z) of 35 to 400. Helium is used as the carrier gas.

Reaction kinetics of isocyanate with α-terpineol: The rate at which the isocyanate reacts with the α-terpineol in the presence of 0.01 wt % DBTL catalyst is analyzed using Fourier-transform infrared (FTIR) spectroscopy. For the FTIR analysis, a Thermo Scientific Nicolet 6700 FTIR spectrometer with ATR (diamond crystal) is used over a frequency range of 400-4,000 cm⁻¹.

### Antifouling Performance.

Bacteria culture and growth: Colonies of Escherichia coli (UTI89) and Staphylococcus aureus (col) are grown overnight at 37° C. onto Lysogeny broth (LB) agar (from Sigma-Aldrich) and Tryptic Soy Agar (TSA, from Sigma-Aldrich), respectively. All colonies are used within two weeks of growth. To perform experiments, one colony of E. coli or S. aureus that is scraped from the LB agar or TSA plate is grown in LB media (Sigma-Aldrich) or tryptic soy broth (1% glucose weight to volume, TSBG, Sigma-Aldrich), respectively, on a ThermoForma orbital shaker un-humidified at 37° C. and 200 rpm. When the optical density (OD) at 600 nm reaches 0.5±0.1 (which is measured with an Ultrospec 2100 pro UV/Visible Spectrophotometer) for E. coli and 0.6±0.1 for S. aureus, respectively, this indicates an approximate concentration of 107 colony forming units (CFU)/mL. The culture is then diluted until the OD reaches 0.02±0.005, representative of about one million CFUs in 100 microliters of culture, and then bacteria are used to test the antifouling capability of the surfaces. Here, the term “colony forming units” is used in place of number of cells because although a cell may be viable, it is not necessarily culturable.

Quantitative culture: The sterilized surfaces are cut to fit the width of the well in a 48 well plate (approximately 0.5 cm by 1 cm) and are placed vertically in the well with approximately one million CFUs total in 1 mL of TSBG. The well plates are then placed on the orbital shaker at 37° C. for 24 hours. On completion, the incubated surfaces are removed from culture, rinsed, and placed in sterile phosphate buffered saline (PBS, Thermo Fisher Scientific). They are then sonicated to remove adhered bacteria from the surface, 7 minutes and 12 minutes for E. coli and S. aureus, respectively, and the acquired solution is then serially diluted in PBS and 10 microliters of each dilution is drop-casted onto TSA plates. The plates are given time to incubate un-humidified at 37° C. overnight and results are then determined by colony enumeration to quantify the number of bacteria adhered to the antifouling polyurethane surfaces.

Contact plate experiments: The sterilized surfaces are cut to an approximate 1 cm×1 cm square and bacteria are grown according to the aforementioned protocol. Once the culture reaches half-log, it is then centrifuged for two minutes at 8000 RPM using a ThermoScientific Sorvall Legend Micro 17R centrifuge. The resulting bacteria pellet is resuspended in 1 ml of 1×PBS, and this rinsing process is carried out two additional times. Finally, the culture is then diluted further and 1 mL of 1×PBS with approximately 10⁶ CFU/mL is pipetted onto each test surface and left in contact for 10 minutes. After the exposure time, the excess liquid is wicked off and dabbed lightly with a sterilized Kimwipe. The exposed surface is then gently placed in contact with agar plates (LB and TSA agar for E. coli and S. aureus, respectively) for one minute. After the contact time, the surface is removed, and the agar plates are incubated un-humidified at 37° C. for 24 hours. Three replicates are tested for each specimen, and results are determined by colony enumeration for each of the samples.

ISO 22196 testing: International Organization for Standardization ISO 22196, Antibacterial Products—Test for Antibacterial Activity and Efficacy is carried out by Microchem Laboratory (Round Rock, TX) with both E. coli (8739) and S. aureus (6538).

Broth culture of wound dressings: The wound dressings are cut 2 cm×1 cm in dimensions and added to an aliquot containing 2 ml of media with approximately 10′ cells of bacteria. Tryptic Soy Broth (TSB) with 1 wt. % glucose is used for MRSA and P. Aeruginosa (PA27853), while Lysogeny broth (LB) is used for E. coli (UTI189). The aliquots are placed in an orbital shaker at 200 rpm at 37° C. for 24 hours. For each independent experiment, triplicate samples are incubated. On completion, the broth corresponding to a replicate is serially diluted in phosphate buffered saline (PBS, Thermo Fischer Scientific) and 10 microliters of each dilution is drop-casted onto TSB or LB agar plates. The plates are given time to incubate un-humidified at 37° C. overnight, and results are then determined by colony enumeration to quantify the number of viable bacteria persisting in the broth.

Instant kill experiment: The experimental and control surfaces are cut to an approximate 1 cm×1 cm square. The bacteria are grown according to the aforementioned protocol. Once the culture reaches half-log, it is then centrifuged for two minutes at 800 RPM using a ThermoScientific Sorvall Legend Micro 17R centrifuge. The resulting bacteria pellet is resuspended in 1 ml of 1×PBS, and this rinsing process is carried out two additional times. Finally, the culture is diluted to 10⁵ and 10⁶ CFUs/10 μl. 10 μl of the suspension is then pipetted onto one face of the sample, and a cover slip is placed on top. The contact time is defined as the time over which the bacterial suspension is in contact with the surface before it is transferred to 2 ml of 1× PBS for quantitative culture.

Fluorescence microscopy: Cells are prepared by the aforementioned protocol. Three microliters of the dye with equal volumes of SYTO® 9 stain and propidium iodide is added to each milliliter of the bacterial suspension. The suspension is incubated at room temperature in the dark for 25 minutes. 10 microliters of the suspension are then pipetted onto the sample and a cover slip is placed over it. Live cells are observed under an FITC filter and dead cells are observed under the Texas Red filter set. A Nikon Eclipse 80i fluorescence microscope and the NIS Elements software are used for imaging.

### Durability Tests.

Linear TABER Abrasion: Mechanical abrasion is performed using a Linear Taber Abrasion machine with a CS-10 resilient abrader and a total weight of 1100 g. The abrader is refaced before each set of abrasion cycles using sand paper (from Taber). Refacing is done at 25 cycles/minute for 25 cycles. For abrasion, samples are clamped down and abraded for up to 1000 cycles at 60 cycles/minute and a stroke length of 50.8 mm. Percent mass loss is calculated over the abraded area.

Environmental exposure: To test the longevity of the antibacterial samples, samples are left in a chemical fume hood, uncovered, with a face velocity of 115 fpm at a 14 inch or 35.56 cm sash height.

Additional abrasion testing: The antimicrobial surface is subjected to different conditions of harsh environments to test durability and longevity of the coating. Mechanical abrasion is performed using a Linear Taber Abrasion machine with a CS-10 resilient abrader and a total weight of 800 g. The abrader is refaced before each set of abrasion cycles using sand paper (from Taber). Refacing is done at 25 cycles/minute for 25 cycles. For abrasion, samples are clamped down and abraded for up to 1000 cycles at 60 cycles/minute and a stroke length of 50.8 mm.

CLOROX® antimicrobial wipe test: An antimicrobial CLOROX® disinfecting wipe is attached to the collet of the Linear Taber Abrasion machine under a total weight of 1.1 kg. The samples are clamped down and wiped for up to 500 cycles at 60 cycles/minute at a stroke length of 50.8 mm. After every 100 cycles, a fresh wipe is installed. The samples are washed with DI water to remove any remnant liquid from the antimicrobial CLOROX® disinfecting wipe before testing for antimicrobial efficacy. A control polyurethane is similarly wiped for comparison.

UV exposure: A sample of DESMOPHEN® polyurethane+35% α-terpineol is placed under 254 nm UVC mercury lamp (UVP, LLC) at a distance of 15 cm. The antimicrobial efficacy is measured after 12 hours of continuous exposure.

Freezing experiment: A sample of DESMOPHEN® polyurethane+35% α-terpineol is placed inside a freezer at −17° C. at 34% R.H. The antimicrobial performance is tested after 25 hours of continuous exposure.

### Results and Discussion

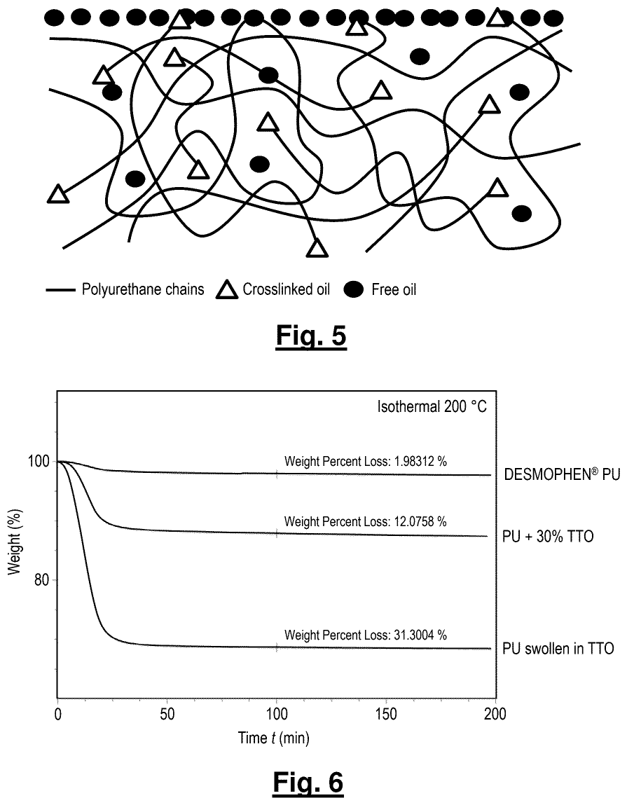

To create antifouling surfaces, the basis of a polyurethane rubber, a diisocyanate plus a polyol, is combined with tea tree oil or eucalyptus oil. Tea tree oil and eucalyptus oil are natural oils comprised of many different organic molecules with compositions that vary depending on where the oil is harvested from and the time of year at which it is harvested. Table 2 shows the compositional makeup of the oils used; about 48% and 91% of the molecules in tea tree oil and eucalyptus oil, respectively, are capable of reacting with the diisocyanate as the polyurethane cross-links, while the rest of the other molecules will most likely not react. Note that the isocyanates can react with the epoxy linkage in eucalyptol. Therefore, the structure of the antifouling polyurethane consists of many polyurethane chains forming the backbone of a polymer network with a partial amount of the oil chemically cross-linked onto a few chain ends and the rest of the oil “free” but stabilized within the network, as shown in FIG. 5. Some, if not most, of the “free” oil assembles at the surface to reduce the overall free energy of the system, adding to the surface's antibacterial capabilities. Since tea tree oil contains fewer molecules with alcohol groups compared to eucalyptus oil, the ratio of “free” oil to cross-linked oil can be higher in tea tree oil than eucalyptus oil.

[symbol]

TABLE 2
Various individual tea tree and eucalyptus oil components and their relative weight
percentages.

| | % in Tea Tree Oil | % in Eucalyptus Oil | |
|---|---|---|---|
| Component | (by weight) | (by weight) | Structure |
| Terpinen-4-ol | 40.6 | — | 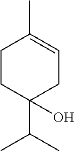 |
| γ-terpinene | 23.6 | 3.45 | 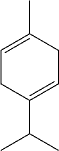 |
| (+)-4-carene | 12.4 | — | 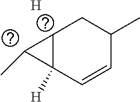 |
| Eucalyptol | 5.05 | 91.0 | 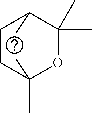 |
| (+)-2-carene | 4.13 | — | 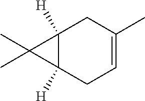 |
| p-cymene | 2.89 | 4.05 | 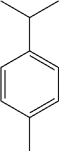 |
| α-pinene | 2.83 | — | 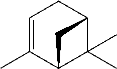 |
| α-terpineol | 2.27 | — | 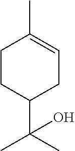 |
| Limonene | 1.29 | — | 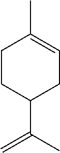 |

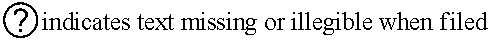

While essential oils are the antibacterial component of the surface, they are very volatile and prone to quick evaporation, making stability a major factor in the surface's design. For one particular system, 30% of tea tree oil (TTO) is reacted into a DESMOPHEN® polyurethane (PU) and then is compared to a pure DESMOPHEN® PU swelled 30% in TTO. FIG. 6 displays the thermogravimetric analysis (TGA) curves for the pure DESMOPHEN® PU, the DESMOPHEN® PU reacted with 30% TTO (PU+30% TTO), and the pure DESMOPHEN® PU swelled in TTO at the 200° C. isotherm. Using the 2% weight loss from the pure DESMOPHEN® PU as a baseline, results show that the swelled DESMOPHEN® PU in TTO loses approximately 29 wt. % while the DESMOPHEN® PU reacted with 30% TTO loses only approximately 10 wt. %. This indicates that the addition of the TTO prior to the PU reaction chemically cross-links in approximately 20% of the TTO, proving an increase in stability compared to the PU simply swelled in TTO. This increases the longevity of the additional 10% of “free” oil, the fraction needed for long-lasting antimicrobial effects.

Using this design, the antibacterial effects of different essential oils and their components are tested within the surfaces against E. coli (gram-negative) and S. aureus (gram-positive) via entire sample incubation with colony enumeration (quantitative culture) and surface incubation with colony enumeration (contact plating). Table 3 outlines every tested surface, with tea tree oil performing better than eucalyptus oil in the DESMOPHEN® PU, even though both oils are antibacterial. This may be due to tea tree oil's higher ratio of “free” to cross-linked oil. Table 3 shows that the lower the number of colony forming units (CFU) on a tested surface, the better their antimicrobial performance. The data is presented as log(CFU). Tested surfaces have a surface area of 100 mm², and at least three replicates are tested for each surface. Since the cross-linked oil stabilizes the “free” oil, it is presumed that the correct ratio of molecules with and without alcohol groups is key to designing an optimized, long-lasting antibacterial surface. It is worth noting, that the addition of tea tree oil into a polydimethyl siloxane (PDMS) network and into an epoxy network, where presumably the oil is only physically cross-linked, also greatly reduces bacteria adhesion at least initially, though these surfaces do not have the same longevity.

TABLE 3
Adhered bacteria per unit surface from quantitive culture experiments conducted
on surfaces with various essential oils and essential oil components.

| | | | E. coli | S. aureus |
|---|---|---|---|---|
| | | % by | UTI89 | col. |
| Polymer Matrix | Component | weight | log(CFU)/ml | log(CFU)/ml |
| Polystyrene | — | — | 6.79 ± 0.41 | 6.53 ± 0.52 |
| DESMOPHEN ® | — | — | 6.68 ± 0.41 | 6.65 ± 0.41 |
| PU | | | | |
| PDMS | — | — | 6.68 ± 0.14 | 6.10 ± 0.00 |
| DESMOPHEN ® | Eucalyptus Oil | 30 | 6.52 ± 0.91 | — |
| PU | Tea Tree Oil | 30 | 3.92 ± 0.46 | 3.35 ± 0.54 |
| | Tea Tree Oil | 30 | 4.64 ± 0.57 | 5.38 ± 0.36 |
| | (Australian) | | | |
| | Tea Tree Oil | 30 | 4.46 ± 0.63 | 4.75 ± 0.76 |
| | (Organic) | | | |
| | Linalool | 15 | — | 6.62 ± 0.12 |
| | α-terpineol | 15 | — | 3.72 ± 0.17 |
| | α-terpineol | 30 | 2.50 ± 0.14 | 0.00 |
| | terpinen-4-ol | 15 | 2.99 ± 0.52 | 7.35 ± 0.56 |
| | γ-terpinene | 10 | — | 7.13 ± 0.13 |
| | p-cymene | 30 | 6.42 ± 0.32 | — |
| | Eucalyptol (50:50) | 30 | 7.38 ± 0.13 | 5.40 ± 0.45 |
| | p-cymene | | | |
| | Eucalyptol (10:90) | 30 | 6.61 ± 0.30 | 4.98 ± 0.76 |
| | p-cymene | | | |
| | Eucalyptol (90:10) | 30 | 6.73 ± 0.45 | 5.70 ± 0.55 |
| | p-cymene | | | |
| | Rosemary Oil | 30 | 5.98 ± 0.65 | 5.43 ± 0.51 |
| PDMS | Tea Tree Oil | 30 | 4.30 ± 0.10 | 3.13 ± 0.17 |
| Epoxy | Tea Tree Oil | 30 | 3.49 ± 0.50 | 5.79 ± 0.81 |

The data in Table 3 shows that tea tree oil obtained from different sources can have different antimicrobial performance. This is likely due to differences in the composition of the oil harvested by different sources and/or at different times of the year. Also, a single component of tea tree oil is identified that shows exceptional antimicrobial performance once cross-linked within a polyurethane. Alpha (α)-terpineol, when cross-linked at 30 wt. % within the polyurethane, leads to a greater than 4-log reduction (4 orders of magnitude) of both E. coli and S. aureus colonies. Cross-linked α-terpineol seems to have the best antimicrobial performance, at least initially, when compared with all compounds tested. Other components of tea tree oil, such as p-cymene, a well-known antimicrobial compound, proved to be ineffective. This is likely because p-cymene cannot react with any of the polyurethane monomers, particularly isocyanates.

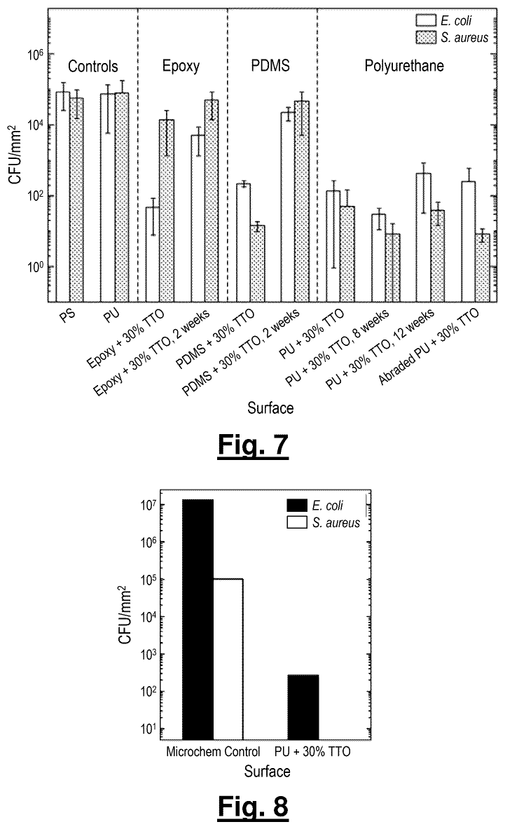

Utilizing polyurethane containing tea tree oil as the optimized surface, FIG. 7 shows the resultant adhered bacteria per unit area from the quantitative culture experiments. When compared against the pure DESMOPHEN® PU, the 30% TTO surface shows a 99.8% and 99.9% reduction with both E. coli and S. aureus, respectively. Even after the samples were left uncovered in a chemical fume hood for 12 weeks, they still display 99% reduction for E. coli and a 99% reduction for S. aureus. In comparison, while epoxy and PDMS surfaces with 30% TTO show an initial reduction in adhered bacteria (at least 99% for both bacteria), after two weeks of chemical fume hood exposure, the surfaces completely foul. This is due to how tea tree oil is only physically mixed into the epoxy and PDMS networks, further validating the requirement of chemically reacting in a fraction of oil to maintain the surface's stability over longer periods of time. This is somewhat of an accelerated test, as there is always a high flow rate of air on top of the surface within the hood. This flow of air will likely increase the evaporation rate of the TTO significantly.

In addition, the DESMOPHEN® PU reacted with 30% TTO surfaces are shipped to an independent, third party laboratory for ISO 22196 testing. FIG. 8 displays the results, with the 30% TTO samples reducing the adhesion of both E. coli and S. aureus, respectively, by 99.998% and greater than 99.995%. The results shown here combined with the results from the official ISO 22196 test foreshadow a long lifetime for the antibacterial capabilities of this surface. In addition, the TTO containing polyurethane can be applied on to any underlying substrate, including different metals, polymers, or glass, by a simple dip, spray, or brush coating.

Quantitative culture experiments are also used to evaluate abraded surfaces that have undergone 1000 abrasion cycles (0.26% mass loss over effectively 100 meters of abrasion) to mimic the wear and tear of everyday use. There is nearly no change in antibacterial properties with a 99.6% and 99.9% reduction of adhered E. coli and S. aureus, respectively, when compared to the pure DESMOPHEN® PU. These results indicate that the antibacterial performance of the surface will not falter even if it is scratched from everyday use.

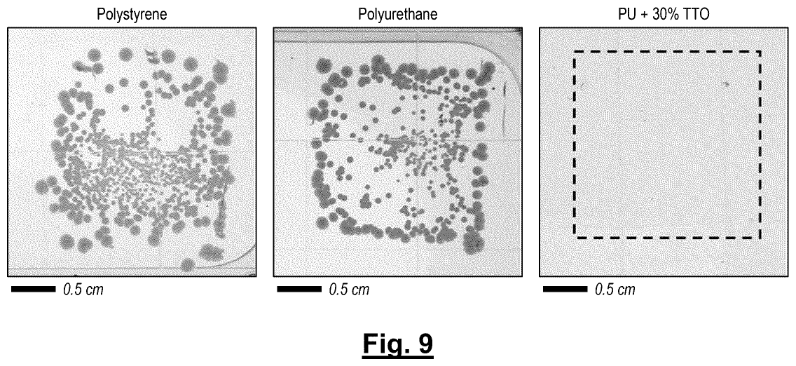

Alongside quantitative culture experiments, contact plating experiments are utilized to investigate how long it takes for the bactericidal properties of the surface to come into effect. FIG. 9 shows the results of these experiments, with the 30% TTO surface showing a greater than 99.99% reduction in E. coli when compared to the pure PU. In just 10 minutes, the surface is capable of killing nearly all the bacteria it is exposed to, demonstrating its rapid effectiveness for real-time applications. Most antimicrobial wipes require a period of action of approximately 10 minutes. Thus, polyurethane surfaces with the cross-linked tea tree oil provide both immediate (microbial death in approximately 10 minutes) and persistent (after 12 weeks) antimicrobial effectiveness. By controlling the fraction of oil cross-linked within the polymer matrix, it is possible to control the immediate and persistent periods of action of the antimicrobial surface. A surface with a higher fraction of unreacted free oil, and less cross-linked oil, will likely require a shorter time to kill the bacteria present on its surface (more immediate), but will not persist over the long term as the antimicrobial oil will evaporate in a shorter time period (less persistent). A surface with a higher fraction of cross-linked oil, and less free oil, will likely demonstrate the opposite behavior, i.e., less immediate but more persistent. It is further likely that this fraction of cross-linked oil would need to be optimized for different applications. An antimicrobial coating for a wound dressing may need to provide more immediate performance (very short kill times), but may only require a time period of action for a few days (less persistent). Other coating, for example, for a cell phone cover, may require more persistent action (over several months), but may only need a bacteria kill time of approximately 30 minutes. Apart from broad spectrum antibacterial properties, tea tree oil also possesses broad spectrum antifungal and antiviral properties. Thus, it is anticipated that the tea tree oil containing polyurethane demonstrated here will similarly display antifungal and antiviral properties.

Because of their unique and pertinent properties, including immediate and persistent antimicrobial effectiveness, durability, and broad spectrum antibacterial, antifungal and antiviral properties, polyurethane-tea tree oil surfaces are ideally suited as antimicrobial coatings on different solid and porous substrates. This approach of cross-linking a portion of the natural oil with a cross-linkable polymer network could similarly be used to fabricate antimicrobial surfaces using other volatile natural oils possessing antimicrobial properties. Such surfaces are expected to have a broad range of applications such as coatings for high-touch areas within hospitals (to reduce hospital acquired infections), daycare facilities, and retirement homes as coatings for sinks, furniture, and wall paint. Applications outside the healthcare space include antimicrobial coatings for touch screens (cell phones, tablets, displays), keyboards, computer mouse, shared automobiles, planes, trains, cruise liners, food contact areas in restaurants, food processing plants, and toilets, for example.

FIG. 10 is a graph showing bacterial growth on everyday surfaces. In particular, the graph shows bacterial growth of MRSA and E. Coli (UTI189) on surfaces of glass, polystyrene (PS), polyurethane (PU), and stainless steel (SS). The initial inoculum was 1 million CFUs, which is depicted by the dotted line. The samples are tested via broth culture over 24 hours at 37° C. inside an orbital shaker (200 RPM).

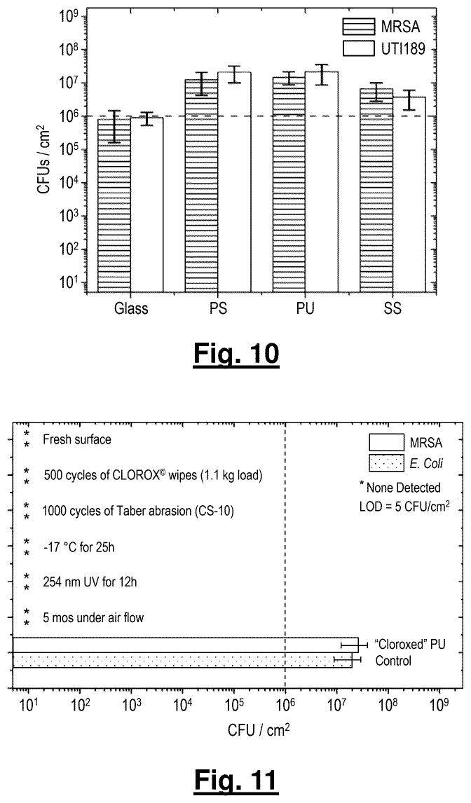

FIG. 11 shows results of durability testing of an antimicrobial coating including a DESMOPHEN® polyurethane polymer matrix and 35 wt. % α-terpineol. The coating is subjected to different durability tests, including 500 cycles of antimicrobial CLOROX® disinfecting wipes, 1000 cycles of linear Taber abrasion, exposure to −17° C. for 25 hours, exposure to 254 nm UVC, and air flow exposure for a duration of 5 months. The samples are tested via broth culture against MRSA and E. Coli over 24 hours at 37° C. inside an orbital shaker (200 RPM). The initial inoculum is 1 million CFUs, which is depicted by the dotted line. A control polyurethane “cloroxed” (i.e., wiped with an antimicrobial CLOROX® disinfecting wipe prior to inoculum exposure) under similar conditions is used as a control. The results show that there was no detectable MRSA or E. coli in any of the antimicrobial coatings tested. Further, the results show that the antimicrobial coatings of the current technology are durable, i.e., they can withstand various types of surface punishments.

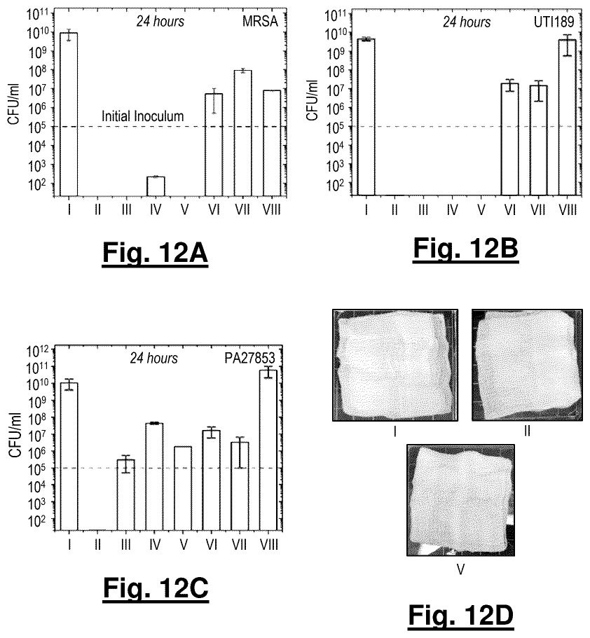

Wound dressings including antimicrobial compositions are also tested, as shown in FIGS. 12A-12D. Composition I is an uncoated gauze, compositions II-V are in accordance with the current technology (each including a matrix of BAYMEDIX® AR602 polyether polyol and BAYMEDIX® AP501 NCO-terminated prepolymer; II having 57 wt. % cinnamaldehyde and 3 wt. % α-terpineol, III having 30 wt. % cinnamaldehyde and 30 wt. % α-terpineol, IV having 60 wt. % α-terpineol, and V having 60 wt. % α-terpineol applied to a thicker 12-ply gauze, VI—VII are commercial antimicrobial dressing controls, and VIII is a control gauze including 0.5 g bacitracin. FIG. 12D shows photographs of dressings I, II, and V. FIG. 12A shows that MRSA was undetectable in dressings II, III, and V and was present well below the initial inoculum level in dressing IV. In contrast, MRSA grew well above the initial inoculum level in dressings I, VI, VII, and VIII. FIG. 12B shows that E. coli was undetectable in dressings II, III, IV, and V. In contrast, E. coli grew well above the initial inoculum level in dressings I, VI, VII, and VIII. FIG. 12C shows the P. Aeruginosa was undetectable in dressing II and was present at levels slightly above the initial inoculum level in dressings III, IV, and V. In contrast, P. Aeruginosa was present at levels above the initial inoculum level in each of dressings I, VI, VII, and VIII. These results show that MRSA and E. coli are sensitive to both cinnamaldehyde and α-terpineol and that P. Aeruginosa is more sensitive to cinnamaldehyde. Therefore, it is shown that the antimicrobial component can be adjusted to be selective against a specific type of bacteria, or the antimicrobial composition can include a plurality of antimicrobial components so that the antimicrobial composition will be effective against a wide range of bacteria types.

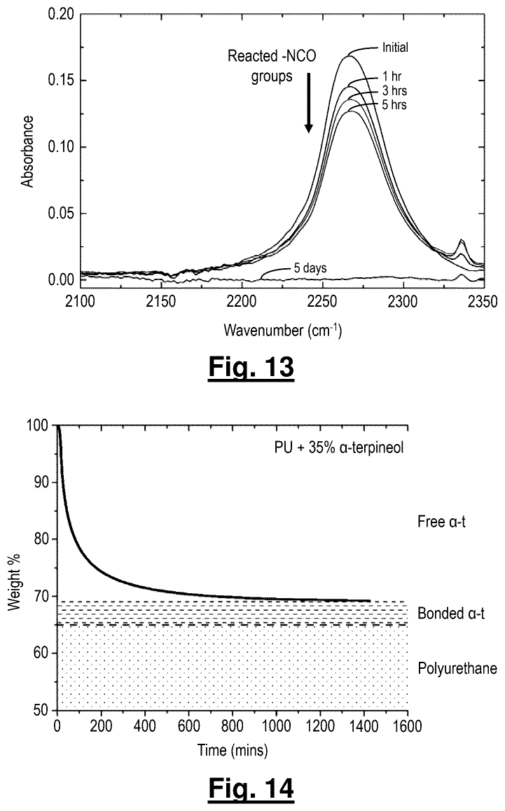

As discussed above, as monomers polymerize in the presence of oil or oil components, increasing amounts of oil components become covalently bonded to the polymer matrix. This is displayed in FIG. 13, which shows reduced absorbance of —NCO peaks over time, indicating that fewer —NCO groups are available for bonding. In other words, as the reaction progresses, the amount of oil components that become covalently bonded to the polymer matrix increases. FIG. 14 shows thermogravimetric analysis isotherms of a antimicrobial composition after reacting for 0-1600 minutes.

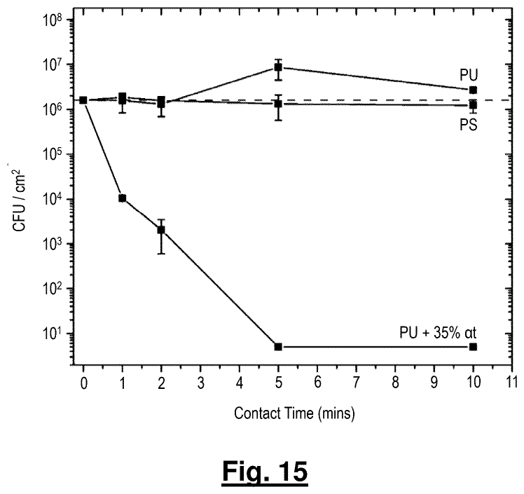

Further tests are performed to show how quickly bacteria are killed by the antimicrobial compositions of the current technology. Using a modified version of ISO 22196, the surface of DESMOPHEN® polyurethane+35% α-terpineol is tested against 10⁶ cells of E. Coli (UTI189 strain). As shown in FIG. 15, a 3-log reduction is observed within the first two minutes. The CFUs reach the limit of detection of 5 CFUs at 5 minutes, showing a 6-log reduction from the initial inoculum of 10⁶ cells (shown by the dotted line). DESMOPHEN® polyurethane and polystyrene are used as control surfaces. This graph shows that the exemplary antimicrobial composition according to the current technology quickly kills bacteria to undetectable levels after about 5 minutes. There is no substantial decrease in bacterial levels in control polymers.

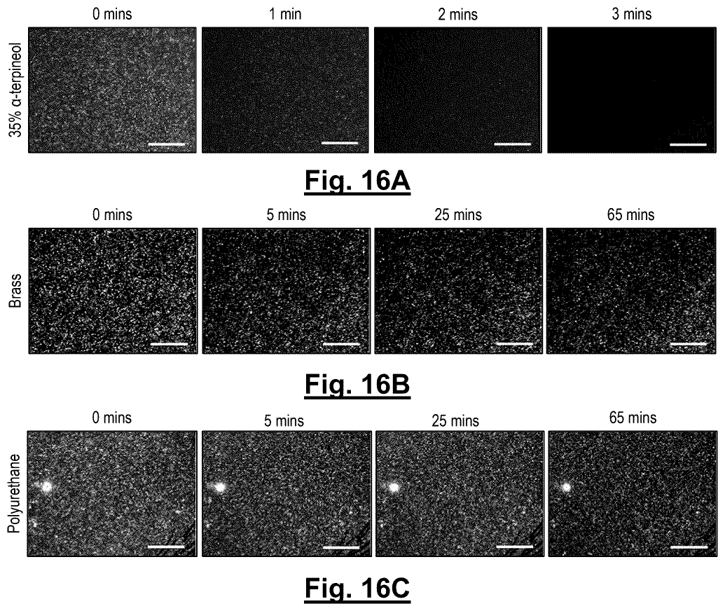

A time-elapsed study of kill performance is also performed using fluorescent E. coli grown on an exemplary antimicrobial composition according to the current technology (DESMOPHEN® polyurethane+35% α-terpineol), brass, and polyurethane using fluorescence microscopy. The results are shown in FIGS. 16A-16C. FIG. 16A shows that after 60 seconds, a large proportion of the fluorescent bacteria have been killed. After 120 seconds, even fewer bacteria are present. After 180 seconds, living bacteria are not detected. In contrast, the levels of bacteria remain constant from 0-66 minutes on brass and polyurethane, as shown in FIGS. 16B-16C.

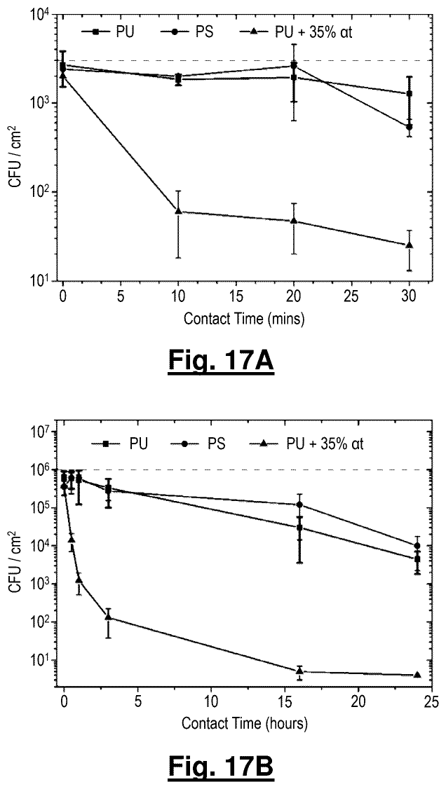

Additional kill performance tests against MRSA are performed using a solid-solid contact plating method. Here, the surface of an exemplary antimicrobial composition according to the current technology (DESMOPHEN® polyurethane+35% α-terpineol) is tested against 3000 cells and 10⁶ cells of MRSA to replicate minor and major contamination events. As shown in FIG. 17A, a 2-log reduction within 10 minutes for the initial inoculum of about 3000 cells (as shown by the dotted line) is observed. As shown in FIG. 17B, upon increasing the initial inoculum to about 10⁶ cells (as shown by the dotted line), a 2-log reduction is observed after 30 minutes. The transfer efficiency is 63.3% for DESMOPHEN® polyurethane, 35.3% for polystyrene and 36.7% for DESMOPHEN® polyurethane+35 wt % α-terpineol. These graphs shows that the exemplary antimicrobial composition according to the current technology quickly kills MRSA in low and high starting levels to undetectable levels. In contrast, there is no substantial decrease in MRSA levels in control polymers.

### CONCLUSIONS

As described above, an antibacterial polymeric surface created by the addition of a volatile natural oil to a cross-linkable polymer before the polymerization of the chain network is shown. The polymeric network is chosen such that it can react with a portion of the chosen antibacterial natural oil. This results in a partial amount of “free” oil stabilized by a fraction of the oil cross-linked into the network, which significantly reduces the evaporation rate of oil from the surface. These results and the results of the ISO 22196 testing indicate a greater than 99% reduction of adhered bacteria on the developed surfaces. Although most of the “free” oil assembles at the surface, it does not quickly evaporate, and even after 12 weeks of exposure to air, the surface shows at least a 99% reduction in adhered bacteria when compared to a polyurethane without the natural oil. This surface is the first of its kind to exhibit exceptional mechanical durability, as demonstrated by its abrasion resistance, and immediate and persistent antimicrobial activity. This approach could similarly be used to fabricate antimicrobial surfaces using other volatile natural oils possessing antimicrobial properties.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for generating an antimicrobial composition, the method comprising:
combining an antimicrobial oil or oil-derived antimicrobial molecules with an uncured polymer precursor solution to form a mixture; and
curing the mixture to generate the antimicrobial composition,
wherein the antimicrobial composition comprises:
a polymer matrix formed from the uncured polymer precursor solution;
an oil-derived component covalently bonded to the polymer matrix; and
an oil-derived antimicrobial component non-covalently associated with at least one of the polymer matrix and the oil-derived component,
wherein the oil-derived component and the oil-derived antimicrobial component are provided from the antimicrobial oil or the oil-derived antimicrobial molecules.

2. The method according to claim 1, wherein the antimicrobial oil is selected from the group consisting of basil oil, bergamot oil, black pepper oil, Brazil's spearmint oil, cardamom oil, cedar oil, cinnamon oil, citron oil, clary sage oil, clove oil, coriander oil, cypress oil, eucalyptus oil, fennel oil, geranium oil, ginger oil, lavender oil, lemongrass oil, mandarin oil, marjoram oil, nutmeg oil, orange oil, oregano oil, palmarosa oil, patchouli oil, peppermint oil, perilla oil, pine oil, rosemary oil, Tahiti lime oil, tea tree oil, thyme oil, vetiver oil, ylang ylang oil, Achillea clavennae, Achillea fragrantissima, Achillea, Achillea ligustica, Artemisia absinthium, Artemisia biennis, Artemisia cana, Artemisia dracunculus, Artemisia longifolia, Artemisia frigida, Cinnamomum zeylancium, Copaifera officinalis, Cuminum cyminum, Cymbopogon citratus, Cymbopogon nardus, Cyperus longus, Daucus littoralis, Dracocephalum foetidum, Eremanthus erythropapps, Eugenia caryophyllata, Euphrasia rostkoviana, Fortunella margarita, Juniperus phoenicea, Laurus nobilis, Juniperus excelsa, Lippia sidoides, Mentha pulegium, Mentha suaveolens, Momordica charantia, Myrtus communis, Nigella sativa, Ocimum gratissimum, Ocimum kilimandscharicum, Origanum vulgare, Ocimum basilicum, Petroselinum sativum, Piper nigrum, Pimpinella anisum, Plectranthus barbatus, P. amboinicus, Plectranthus neochilus, Pogostemon cablin, Rosmarinus officinalis, Satureja hortensis, Salvia officinalis, Salvia lavandulifolia, Satureja cuneifolia, Struchium sparganophora, Syzygium cumini, Trachyspermum ammi, Thymus zygis, Thymus mastichina, Thymus kotschyanus, Thuja sp. (Thuja plicata, Thuja occidentalis), Verbena officinalis, Warionia saharae, fractions thereof, components thereof, molecules thereof, and combinations thereof.

3. The method according to claim 1, wherein the antimicrobial oil is tea tree oil, eucalyptus oil, or a combination thereof.

4. The method according to claim 1, wherein the oil-derived antimicrobial molecules are selected from the group consisting of alkaloids, glycosides, terpenes, terpenoids, isoprenoids, saponins, steroids, flavonoids, isoflavonoids, phenolics, polyphenols, phenylpropanoids, phenylpropenes, coumarins, curcuminoids, and combinations thereof.

5. The method according to claim 1, wherein the uncured polymer precursor solution comprises at least one monomer.

6. The method according to claim 5, wherein the curing includes covalently bonding the oil-derived component to a portion of the at least one monomer and polymerizing a remaining portion of the at least one monomer to form the polymer matrix with the oil-derived component covalently bonded thereto.

7. The method according to claim 1, wherein the uncured polymer precursor solution comprises either diisocyanate or dicarboxylic acid and polyol.

8. The method according to claim 1, wherein the antimicrobial composition is a film and the method further comprises:
disposing an adhesive onto a surface of the film.

9. The method according to claim 1, wherein the method is performed on either a high-touch surface or on a medical implant.

10. The method according to claim 1, further comprising:
disposing a wound dressing into the mixture; and
performing the curing while the wound dressing is disposed in the mixture,
wherein, after the curing, an antimicrobial wound dressing comprising the antimicrobial composition is formed.

11. The method according to claim 1, wherein the mixture comprises greater than or equal to about 1 wt. % to less than or equal to about 95 wt. % of the antimicrobial oil.

12. The method according to claim 1, wherein the method further comprises selecting the antimicrobial oil and the uncured polymer precursor solution such that the antimicrobial composition has a desired potency or effective duration.

13. The method according to claim 1, wherein the polymer matrix comprises polyurethane, the antimicrobial oil is an essential oil-derived component comprising at least one hydroxy or epoxy group covalently bonded within the polymer matrix to form cross-links therein, wherein the polymer matrix having the oil-derived component covalently bonded therein is formed by reacting a first monomer comprising an isocyanate, a second monomer comprising a polyol, and the oil-derived component is an essential-oil derived component comprising at least one hydroxy or epoxy group together, so that the hydroxy or epoxy group reacts with the isocyanate to form covalent bonds during polymerization of the first monomer and the second monomer; and
the antimicrobial composition has greater than a 2-log reduction of an adhered bacteria after exposure for both 10 minutes and after 1 week, as compared with a polymeric matrix lacking the essential oil-derived component and the oil-derived antimicrobial component.

14. The method according to claim 1, wherein the oil-derived component comprises alpha-terpineol, and the oil-derived antimicrobial component comprises alpha-terpineol.

15. The method according to claim 1, wherein the oil-derived component and the oil-derived antimicrobial component are present in a ratio of the oil-derived component:oil-derived antimicrobial component of from about 1:3 to about 3:1.

16. A method of preparing an antimicrobial surface, the method comprising:
applying a mixture onto a surface, the mixture comprising an antimicrobial oil or oil-derived antimicrobial molecules and an uncured polymer precursor solution; and
incubating the mixture on the surface until the mixture cures and forms an antimicrobial film on the surface, the antimicrobial film comprising:
a polymer matrix formed from the uncured polymer precursor solution;
an oil-derived component covalently bonded to the polymer matrix; and
an oil-derived antimicrobial component non-covalently associated with at least one of the polymer matrix and the oil-derived component,
wherein the oil-derived component and the oil-derived antimicrobial component are provided from the antimicrobial oil or the oil-derived antimicrobial molecules.

17. The method according to claim 16, wherein the antimicrobial oil is a natural oil extracted from a plant.

18. The method according to claim 16, wherein the mixture is composed from a kit comprising:
at least one uncured monomer;
the antimicrobial oil or the oil-derived antimicrobial molecules; and
optionally at least one of an initiator and an activator.

19. The method according to claim 16, wherein the antimicrobial film has a thickness of greater than or equal to about 1 μm to less than or equal to about 10 mm.

20. The method according to claim 16, wherein the surface on which the mixture is applied is a high-touch surface selected from the group consisting of a counter, a toilet, a sink, flooring, tiles, a dashboard, a handhold, a handle, a door handle, a door knob, a handrail, a cup holder, a touch screen, a tray, a tray table, furniture, paint, a table, a chair, a seat, a fabric, a gear shifter, and a steering wheel.

21. The method according to claim 16, wherein the surface on which the mixture is applied is a surface of a medical implant or a surface of a wound dressing.

22. A method of rejuvenating an antimicrobial surface prepared by the method according to claim 16, the method comprising:
applying a fresh antimicrobial oil to the antimicrobial surface; and
incubating the antimicrobial surface until the fresh antimicrobial oil becomes physically associated with at least one of the antimicrobial film and the polymer matrix.

23. A method for generating an antimicrobial composition, the method comprising:
combining an antimicrobial oil or oil-derived antimicrobial molecules having at least one hydroxy or epoxy group with an uncured polymer precursor solution comprising a first monomer comprising an isocyanate and a second monomer comprising a polyol to form a mixture; and
reacting the first monomer, the second monomer, and the antimicrobial oil or oil-derived antimicrobial molecules in the mixture so that the at least one hydroxy or epoxy group of the antimicrobial oil or oil-derived antimicrobial molecules reacts with the isocyanate to form covalent bonds during polymerization to generate the antimicrobial composition,
wherein the antimicrobial composition comprises:
a polymer matrix comprising polyurethane is formed from the uncured polymer precursor solution;
an essential oil-derived component covalently bonded within the polymer matrix to form cross-links therein; and
an oil-derived antimicrobial component non-covalently associated with at least one of the polymer matrix and the essential oil-derived component,
wherein the essential oil-derived component and the oil-derived antimicrobial component are provided from the antimicrobial oil or the oil-derived antimicrobial molecules, wherein the antimicrobial composition has greater than a 2-log reduction of an adhered bacteria after exposure for both 10 minutes and after 1 week, as compared with a polymeric matrix lacking the essential oil-derived component and the oil-derived antimicrobial component.

24. The method according to claim 23, wherein the oil-derived antimicrobial molecules are selected from the group consisting of alkaloids, glycosides, terpenes, terpenoids, isoprenoids, saponins, steroids, flavonoids, isoflavonoids, phenolics, polyphenols, phenylpropanoids, phenylpropenes, coumarins, curcuminoids, and combinations thereof.

25. The method according to claim 23, wherein the reacting includes covalently bonding the essential oil-derived component to the first monomer and polymerizing a remaining portion of the first monomer and second monomer to form the polymer matrix with the essential oil-derived component covalently bonded thereto.

26. The method according to claim 23, wherein the oil-derived component comprises alpha-terpineol, and the oil-derived antimicrobial component comprises alpha-terpineol.

27. The method according to claim 23, wherein the oil-derived component and the oil-derived antimicrobial component are present in a ratio of the oil-derived component:oil-derived antimicrobial component of from about 1:3 to about 3:1.
